(19) United States
(12) Patent Application Publication
(10) Pub. No.: US 2017/0166968 A1
(43) Pub. Date: Jun. 15, 2017

(54) METHODS FOR DIAGNOSING AND TREATING NEUROIMMUNE-BASED PSYCHIATRIC DISORDERS

(71) Applicant: THE REGENTS OF THE UNIVERSITY OF CALIFORNIA, Oakland, CA (US)

(72) Inventors: A. Kimberley McAllister, Davis, CA (US); Myka E. Estes, Davis, CA (US); Melissa Bauman, Davis, CA (US); Cameron Carter, Davis, CA (US)

(73) Assignee: THE REGENTS OF THE UNIVERSITY OF CALIFORNIA, Oakland, CA (US)

(21) Appl. No.: 15/342,900

(22) Filed: Nov. 3, 2016

Related U.S. Application Data
(63) Continuation of application No. PCT/US2015/029407, filed on May 6, 2015.
(60) Provisional application No. 61/989,791, filed on May 7, 2014.

(51) Int. Cl. C12Q 1/68

(52) U.S. Cl. CPC C12Q 1/6883; C12Q 2600/118; C12Q 2600/158; C12Q 2600/136

(57) ABSTRACT

The methods of the present invention are useful for determining whether an individual has or is at risk of developing a neuroimmune-based psychiatric disorder by detecting the expression level of one or more cytokine receptors in a biological sample. The methods of the present invention are also useful for the in vivo imaging of brain tissue by detecting one or more imaging agents such as ligands (e.g., cytokines) that bind to one or more cytokine receptors. In addition, the methods of the present invention are useful for identifying compounds that modulate (e.g., increase) the expression level or activity of one or more cytokine receptors. The present invention further provides therapeutic methods that target one or more cytokine receptors for the treatment of a neuroimmune-based psychiatric disorder.

### CROSS-REFERENCES TO RELATED APPLICATIONS

The present application is a continuation of International Application No. PCT/US2015/029407, filed May 6, 2015, which claims priority to U.S. Provisional Application No. 61/989,791, filed May 7, 2014, the contents of which are herein incorporated by reference in their entirety for all purposes.

### BACKGROUND OF THE INVENTION

Psychiatric disorders such as schizophrenia, major depressive disorder, and bipolar disorder are a major public health problem, affecting a significant portion of the adult population of the United States each year. For example, schizophrenia is a chronic and debilitating psychiatric disorder that affects approximately 1% of the population. While it has been hypothesized that such disorders have genetic roots, little progress has been made in identifying gene sequences and gene products that play a role in causing these disorders, as is true for many diseases with a complex genetic origin (see, e.g., Burmeister, Biol. Psychiatry, 45:522-532 (1999)).

In recent years, the role of a dysregulated immune system has been implicated in the etiology of a number of psychiatric disorders. Studies have demonstrated that some patients with psychiatric disorders exhibit characteristic signs of an altered immune system, which may be a common pathophysiological mechanism that underlies the development and progression of these disorders. For example, evidence suggests that immune dysregulation is associated with the neurobiological etiology of autism spectrum disorders and major depressive disorder. In particular, findings of abnormal immune responses, neuroinflammation, and microglial activation, and the presence of maternal autoantibodies to fetal brain tissue have been described for autism spectrum disorders (see, e.g., Vargas et al., Ann Neurol., 57:67-81 (2005); Singer et al., J Neuroimmunol., 211:39-48 (2009)). Schizophrenia patients also exhibit immunological abnormalities. In fact, maternal infection and the subsequent inflammatory response during pregnancy is considered to be a high risk factor for the development of schizophrenia and other related disorders in the offspring. However, there is currently no effective way to determine whether an individual has or is at risk of developing a neuroimmune-based psychiatric disorder. Similarly, there is a need in the art for therapeutic agents capable of targeting the gene sequences or gene products that play a role in causing these disorders. The present invention satisfies these needs and provides related advantages as well.

### BRIEF SUMMARY OF THE INVENTION

The methods of the present invention are useful for determining whether an individual has or is at risk of developing a neuroimmune-based psychiatric disorder by detecting the expression level of one or more cytokine receptors in a biological sample. The methods of the present invention are also useful for the in vivo imaging of brain tissue by detecting one or more imaging agents such as ligands (e.g., cytokines) that bind to one or more cytokine receptors. In addition, the methods of the present invention are useful for identifying compounds that modulate (e.g., increase) the expression level or activity of one or more cytokine receptors. The present invention further provides therapeutic methods that target one or more cytokine receptors for the treatment of a neuroimmune-based psychiatric disorder.

In one aspect, the present invention provides a method for determining whether an individual has or is at risk of developing a neuroimmune-based psychiatric disorder, the method comprising:
(a) detecting in a biological sample from the individual the expression level of one or more cytokine receptors selected from the group consisting of CR3, CCR5, CX3CR1, IL1R, IL3R, GM-CSFR, and combinations thereof;
(b) comparing the expression level of the one or more cytokine receptors detected in the biological sample to a control expression level of the one or more cytokine receptors; and
(c) determining that the individual has or is at risk of developing a neuroimmune-based psychiatric disorder when the expression level of the one or more cytokine receptors detected in the biological sample is decreased compared to a control expression level of the one or more cytokine receptors.

In another aspect, the present invention provides a method for the in vivo imaging of brain tissue for determining whether an individual has or is at risk of developing a neuroimmune-based psychiatric disorder, the method comprising:
(a) administering to the individual one or more imaging agents comprising one or more ligands that bind to one or more cytokine receptors selected from the group consisting of CR3, CCR5, CX3CR1, IL1R, IL3R, GM-CSFR, and combinations thereof, wherein a detectable moiety is attached to the one or more ligands; and
(b) detecting the one or more imaging agents in brain tissue of the individual, wherein the individual has or is at risk of developing a neuroimmune-based psychiatric disorder when the level of the one or more imaging agents detected in the frontal cortex is less than the level of the one or more imaging agents detected in an individual or a population of individuals without the neuroimmune-based psychiatric disorder.

In yet another aspect, the present invention provides a method for identifying a compound for treating a neuroimmune-based psychiatric disorder, the method comprising:
(a) contacting the compound with one or more cytokine receptors selected from the group consisting of CR3, CCR5, CX3CR1, IL1R, IL3R, GM-CSFR, and combinations thereof; and
(b) determining whether the compound increases the expression level or activity of the one or more cytokine receptors, thereby identifying a compound for treating a neuroimmune-based psychiatric disorder.

In a further aspect, the present invention provides a method for treating a neuroimmune-based psychiatric disorder in an individual in need thereof, the method comprising:
(a) administering to the individual a therapeutically effective amount of a compound identified using the method described herein.

In a related aspect, the present invention provides a method for treating a neuroimmune-based psychiatric disorder in an individual in need thereof, the method comprising:
(a) administering to the individual a therapeutically effective amount of a nucleic acid encoding one or more cytokine receptors selected from the group consisting of CR3, CCR5, CX3CR1, IL1R, IL3R, GM-CSFR, and combinations thereof.

Other objects, features, and advantages of the present invention will be apparent to one of skill in the art from the following detailed description and figures.

### BRIEF DESCRIPTION OF THE DRAWINGS

FIG. 1 illustrates the results of gene expression profiling of candidate genes from (A) mouse and (B) non-human primate (NHP) RNA samples following maternal immune activation (MIA).

FIG. 2 provides a summary of the MIA-induced changes in cytokine receptors in the frontal cortex in both mouse and NHP offspring.

FIG. 3 provides a summary of changes in cytokine receptors in the frontal cortex of MIA offspring from the mouse model at 5 postnatal ages.

### DETAILED DESCRIPTION OF THE INVENTION

### I. INTRODUCTION

There is increasing evidence that immune dysregulation may underlie several psychiatric and neurological disorders, yet there is currently no effective way to determine which individuals have a neuroimmune basis for their disease. The only approach to image neural inflammation in the brain in vivo is to use a PET tracer called PK11195, an isoquinoline carboxamide which binds to the peripheral benzodiazepine receptor (PBR). However, this tracer is less than optimal due to its low signal to noise and lack of specificity for any particular cytokine pathway. Since there are over one hundred different cytokines and corresponding receptors in humans, selecting which one to target for developing novel diagnostic tools and/or therapies for psychiatric disorders is extremely challenging and difficult. The use of animal models of psychiatric disorders is important for identifying potential targets, but mouse models are not always accurate predictors of human neurobiology. Thus, the methods of the present invention are particularly advantageous because they are based on the use of a novel approach to identify and select molecular targets that are altered in both the mouse and non-human primate (NHP) brains in animal models for schizophrenia and autism spectrum disorder (i.e., maternal immune activation (MIA) model). Example 1 describes a study using this approach to identify a set of cytokine receptors that form the basis for the targeted development of diagnostic methods, imaging tools, screening methods, and therapeutic agents for neuroimmune-based psychiatric disorders.

In particular, the present invention is based, in part, on the discovery that several cytokines and their receptors are altered in the brains of postnatal offspring following MIA. There are many cytokine receptors whose expression is altered in the brains of MIA offspring from the mouse model at multiple ages. Notably, the discovery of a subset of these receptors that are also altered in the brains of MIA offspring from the NHP model is highly significant because (1) this is the only NHP MIA model in the world that has undergone this analysis and (2) the conservation of MIA-induced changes in a subset of cytokine receptors in the brains across such divergent species as the mouse and NHP indicate that these cytokine receptors are likely to be essential for the MIA phenotype in these offspring and for similar phenotypes in psychiatric disorders with a neuroimmune basis. Because maternal infection is a risk factor for both schizophrenia and autism spectrum disorder, these cytokine pathways may serve as targets for the development of novel diagnostic tools and screens for new treatments.

### II. DEFINITIONS

The terms “a,” “an,” or “the” as used herein not only include aspects with one member, but also include aspects with more than one member. For instance, the singular forms “a,” “an,” and “the” include plural referents unless the context clearly dictates otherwise. Thus, for example, reference to “a cell” includes a plurality of such cells and reference to “the agent” includes reference to one or more agents known to those skilled in the art, and so forth.

The terms “subject”, “patient” or “individual” are used herein interchangeably to refer to a human or animal. For example, the animal subject may be a mammal, a primate (e.g., a monkey), a livestock animal (e.g., a horse, a cow, a sheep, a pig, or a goat), a companion animal (e.g., a dog, a cat), a laboratory test animal (e.g., a mouse, a rat, a guinea pig, a bird), an animal of veterinary significance, or an animal of economic significance.

A “psychiatric disorder” or “psychiatric illness” includes mood disorders (e.g., depression of all forms and/or types, bipolar disorder, etc.), schizophrenia, autism spectrum disorder, personality disorders, anxiety, anxiety disorders, substance-related disorders, childhood disorders, dementia, adjustment disorder, delirium, multi-infarct dementia, and Tourette's disorder as described in, e.g., the Diagnostic and Statistical Manual (DSM) of Mental Disorders, Fifth Edition (DSM-5). Typically, such disorders have a complex genetic, biochemical, and/or environmental component.

A “mood disorder” includes disruption of feeling, tone or emotional state experienced by an individual for an extensive period of time. Mood disorders include, but are not limited to, depression (i.e., depressive disorders), bipolar disorders, substance-induced mood disorders, alcohol-induced mood disorders, benzodiazepine-induced mood disorders, mood disorders due to general medical conditions, as well as many others. See, e.g., DSM-5.

The term “depression” or “depressive disorder” refers to a mood disorder involving any of the following symptoms: persistent sad, anxious, and/or “empty” mood; feelings of hopelessness and/or pessimism; feelings of guilt, worthlessness, and/or helplessness; loss of interest or pleasure in hobbies and activities that were once enjoyed, including sex; decreased energy, fatigue, and/or being “slowed down”; difficulty concentrating, remembering, and/or making decisions; insomnia, early-morning awakening, and/or oversleeping; loss of appetite and/or weight loss, overeating and/or weight gain; thoughts of death and/or suicide; suicide attempts; restlessness and/or irritability; persistent physical symptoms that do not respond to treatment, such as headaches, digestive disorders, and/or chronic pain; and combinations thereof. See, e.g., DSM-5. Non-limiting examples of depressive disorders include major depression disorder (MDD), atypical depression, melancholic depression, psychotic major depression or psychotic depression, catatonic depression, postpartum depression, seasonal affective disorder (SAD), chronic depression (dysthymia), double depression, depressive disorder not otherwise specified, depressive personality disorder (DPD), recurrent brief depression (RBD), minor depressive disorder (minor depression), premenstrual syndrome, premenstrual dysphoric disorder, depression caused by chronic medical conditions (e.g., cancer, chronic pain, chemotherapy, chronic stress), and combinations thereof. Various subtypes of depression are described in, e.g., DSM-5. In particular embodiments, the depression is major depressive disorder (MDD).

“Bipolar disorder” includes a mood disorder characterized by alternating periods of extreme moods. A person with bipolar disorder experiences cycling of moods that usually swing from being overly elated or irritable (mania) to sad and hopeless (depression) and then back again, with periods of normal mood in between. Diagnosis of bipolar disorder is described in, e.g., DSM-5. Bipolar disorders include bipolar disorder I (mania with or without major depression), bipolar disorder II (hypomania with major depression), and cyclothymia. See, e.g., DSM-5. Bipolar disorder is also known as manic depression.

“Schizophrenia” refers to a psychiatric disorder involving a withdrawal from reality by an individual. Symptoms comprise for at least a part of a month two or more of the following symptoms: delusions (only one symptom is required if a delusion is bizarre, such as being abducted in a space ship from the sun); hallucinations (only one symptom is required if hallucinations are of at least two voices talking to one another or of a voice that keeps up a running commentary on the patient's thoughts or actions); disorganized speech (e.g., frequent derailment or incoherence); grossly disorganized or catatonic behavior; or negative symptoms, i.e., affective flattening, alogia, or avolition. Schizophrenia encompasses disorders such as, e.g., schizoaffective disorders. Diagnosis of schizophrenia is described in, e.g., DSM-5. Types of schizophrenia include, e.g., paranoid, disorganized, catatonic, undifferentiated, and residual. See, e.g., DSM-5.

The terms “autism spectrum disorder,” “autistic spectrum disorder,” “autism” or “ASD” interchangeably refer to a spectrum of neurodevelopmental disorders characterized by impaired social interaction and communication accompanied by repetitive and stereotyped behavior. Autism includes a spectrum of impaired social interaction and communication; however, the disorder can be roughly categorized into “high functioning autism” or “low functioning autism,” depending on the extent of social interaction and communication impairment. Individuals diagnosed with “high functioning autism” have minimal but identifiable social interaction and communication impairments (e.g., Asperger's syndrome). Additional information on autism spectrum disorders can be found in, e.g., DSM-5; Autism Spectrum Disorders: A Research Review for Practitioners, Ozonoff et al., eds., 2003, American Psychiatric Pub; Gupta, Autistic Spectrum Disorders in Children, 2004, Marcel Dekker Inc; Hollander, Autism Spectrum Disorders, 2003, Marcel Dekker Inc; Handbook of Autism and Developmental Disorders, Volkmar, ed., 2005, John Wiley; Sicile-Kira and Grandin, Autism Spectrum Disorders: The Complete Guide to Understanding Autism, Asperger's Syndrome, Pervasive Developmental Disorder, and Other ASDs, 2004, Perigee Trade; and Duncan et al., Autism Spectrum Disorders [Two Volumes]: A Handbookfor Parents and Professionals, 2007, Praeger.

The term “neuroimmune-based psychiatric disorder” includes a psychiatric disorder in which an altered or dysregulated immune system underlies the development and/or progression of the disorder. Non-limiting examples of neuroimmune-based psychiatric disorders include mood disorders such as depression (e.g., major depressive disorder) and bipolar disorder, schizophrenia, and autism spectrum disorder.

The term “biological sample” refers to any sample comprising biological material from any biological source that may contain an analyte (e.g., cytokine receptor) of interest. For example, “biological sample” may include whole blood, serum, plasma, saliva, urine, cerebrospinal fluid, amniotic fluid, nipple aspirate, feces, bile, tears, perspiration, sperm, vaginal fluid, or tissue sample (e.g., brain tissue). In some embodiments, the biological sample is derived, e.g., by biopsy, from cells, tissues, or organs. In certain instances, the biological sample is a tissue sample from a specific brain region such as the frontal cortex (e.g., dorsolateral prefrontal cortex (DLPFC)).

The term “ligand that binds to a cytokine receptor” refers to the binding/interaction of a peptide motif in a ligand (e.g., a binding reagent such as a cytokine, an antibody specific for the cytokine receptor, a fragment thereof, and the like), which shows the capacity of specific interaction with a specific cytokine receptor or a specific group of cytokine receptors. In certain embodiments, the term refers to the ability of a ligand or a portion thereof to interact with and/or bind to a target cytokine receptor and without cross-reacting with molecules of similar sequences or structures. In some instances, a ligand specifically binds to a target cytokine receptor when it binds to the target cytokine receptor with a substantially lower dissociation constant (i.e., tighter binding) than a molecule of similar sequence or structure. For example, in certain instances, a specific binding occurs when the ligand binds to the target cytokine receptor with an about 2, 3, 4, 5, 6, 8, 10, 15, 20, 25, 30, 40, 50, 100, or 1000-fold or greater affinity than a related molecule. The binding of the ligand to a site on the target cytokine receptor may occur via intermolecular forces such as ionic bonds, hydrogen bonds, hydrophobic interactions, dipole-dipole bonds, and/or Van der Waals forces. Cross-reactivity may be tested, for example, by assessing binding of the ligand under conventional conditions to the target cytokine receptor as well as to a number of more or less (e.g., structurally and/or functionally) closely related molecules. These methods may include, without limitation, binding studies, blocking and competition studies with closely related molecules, FACS analysis, surface plasmon resonance (e.g., with BIAcore), analytical ultracentrifugation, isothermal titration calorimetry, fluorescence anisotropy, fluorescence spectroscopy, radiolabeled ligand binding assays, and combinations thereof.

“Inhibitors,” “activators,” and “modulators” of binding or activity include inhibitory, activating, or modulating molecules, respectively, identified using in vitro and in vivo assays for binding or activity, e.g., ligands, agonists, antagonists, homologs, and mimetics thereof. The term “modulator” includes inhibitors and activators. Inhibitors are agents that, e.g., bind to a polypeptide and inhibit, partially or totally block stimulation or enzymatic activity, decrease, prevent, delay activation, inactivate, desensitize, or downregulate the activity or expression of the polypeptide, e.g., antagonists. Activators are agents that, e.g., bind to, stimulate, increase, open, activate, facilitate, enhance activation or enzymatic activity, sensitize or upregulate the activity or expression of a polypeptide, e.g., agonists. Modulators include naturally-occurring and synthetic ligands, antagonists, agonists, small chemical molecules and the like.

The term “test compound” or “drug candidate” includes any molecule, either naturally-occurring or synthetic, e.g., protein, polypeptide, peptide, small organic molecule, polysaccharide, lipid, fatty acid, polynucleotide, oligonucleotide, etc. The test compound can be in the form of a library of test compounds, such as a combinatorial or randomized library that provides a sufficient range of diversity. Test compounds are optionally linked to a fusion partner, e.g., targeting compounds, rescue compounds, dimerization compounds, stabilizing compounds, addressable compounds, and other functional moieties. Conventionally, new chemical entities with useful properties are generated by identifying a test compound (called a “lead compound”) with some desirable property or activity, e.g., inhibiting activity, creating variants of the lead compound, and evaluating the property and activity of those variant compounds. In certain embodiments, high throughput screening (HTS) methods are employed for such an analysis.

The term “isolated,” when applied to a nucleic acid or protein, denotes that the nucleic acid or protein is essentially free of other cellular components with which it is associated in the natural state. It is preferably in a homogeneous state although it can be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified. In particular, an isolated gene is separated from open reading frames that flank the gene and encode a protein other than the gene of interest. The term “purified” denotes that a nucleic acid or protein gives rise to essentially one band in an electrophoretic gel. Particularly, it means that the nucleic acid or protein is at least 85% pure, at least 95% pure, or at least 99% pure.

The term “nucleic acid” or “polynucleotide” includes deoxyribonucleotides or ribonucleotides and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Cassol et al. (1992); Rossolini et al., Mol. Cell. Probes 8:91-98 (1994)). The term nucleic acid is used interchangeably with gene, cDNA, and mRNA encoded by a gene.

The terms “polypeptide,” “peptide,” and “protein” are used interchangeably herein to include a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymers. As used herein, the terms encompass amino acid chains of any length, including full-length proteins (i.e., antigens), wherein the amino acid residues are linked by covalent peptide bonds.

The term “amino acid” includes naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs include compounds that have the same basic chemical structure as a naturally occurring amino acid, i.e., an α carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. “Amino acid mimetics” include chemical compounds that have a structure that is different from the general chemical structure of an amino acid, but that functions in a manner similar to a naturally occurring amino acid.

Amino acids may be referred to herein by either the commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, “conservatively modified variants” include those nucleic acids that encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein that encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid that encodes a polypeptide is implicit in each described sequence.

As to amino acid sequences, one of skill will recognize that individual substitutions, deletions and/or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and/or alleles.

The following eight groups each contain amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine (S), Threonine (T); and
8) Cysteine (C), Methionine (M)
   (see, e.g., Creighton, Proteins (1984)).

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. For example, recombinant cells express genes that are not found within the native (non-recombinant) form of the cell or express native genes that are otherwise abnormally expressed, under-expressed, or not expressed at all.

The term “heterologous” when used with reference to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein comprises two or more subsequences that are not found in the same relationship to each other in nature (e.g., a fusion protein).

The phrase “nucleic acid encoding” refers to a nucleic acid that contains sequence information for a structural RNA such as rRNA or tRNA, or the primary amino acid sequence of a specific protein, polypeptide, or peptide, or a binding site for a trans-acting regulatory agent. This phrase specifically encompasses degenerate codons (i.e., different codons which encode a single amino acid) of the native sequence or sequences which may be introduced to conform with codon preference in a specific host cell.

An “expression vector” includes a nucleic acid construct, generated recombinantly or synthetically, with a series of specified nucleic acid elements that permit transcription of a particular nucleic acid in a host cell. The expression vector can be part of a plasmid, virus, or nucleic acid fragment. Typically, the expression vector includes a nucleic acid to be transcribed operably linked to a promoter.

An individual who is “at risk of developing a neuroimmune-based psychiatric disorder” refers to an individual (e.g., a human) who has an inclination or a higher likelihood of developing a neuroimmune-based psychiatric disorder when compared to an average individual (e.g., a human) in the general or control population.

A “therapeutically effective amount” includes an amount or quantity effective, at dosages and for periods of time necessary, to achieve the desired therapeutic or prophylactic result.

### III. DETAILED DESCRIPTION OF THE EMBODIMENTS

In one aspect, the present invention provides a method for determining whether an individual has or is at risk of developing a neuroimmune-based psychiatric disorder, the method comprising:
(a) detecting in a biological sample from the individual the expression level of one or more cytokine receptors selected from the group consisting of CR3, CCR5, CX3CR1, IL1R, IL3R, GM-CSFR, and combinations thereof;
(b) comparing the expression level of the one or more cytokine receptors detected in the biological sample to a control expression level of the one or more cytokine receptors; and
(c) determining that the individual has or is at risk of developing a neuroimmune-based psychiatric disorder when the expression level of the one or more cytokine receptors detected in the biological sample is decreased compared to a control expression level of the one or more cytokine receptors.

In some embodiments, the neuroimmune-based psychiatric disorder is schizophrenia (SZ) or an autism spectrum disorder (ASD). In other embodiments, the neuroimmune-based psychiatric disorder is a mood disorder such as, e.g., major depressive disorder (MDD) or bipolar disorder. In preferred embodiments, the individual is a human.

In certain embodiments, the expression level of any one or a plurality (e.g., at least two, three, four, five, or six) of the cytokine receptors is detected. In particular embodiments, the expression level of one, two, three, or all four cytokine receptors selected from the group consisting of CR3, CCR5, CX3CR1, and IL1R is detected.

In some instances, the expression level of CR3 and CCR5 is detected. In other instances, the expression level of CR3 and CX3CR1 is detected. In some instances, the expression level of CR3 and IL1R is detected. In other instances, the expression level of CR3 and IL3R is detected. In some instances, the expression level of CR3 and GM-CSFR is detected. In other instances, the expression level of CCR5 and CX3CR1 is detected. In some instances, the expression level of CCR5 and IL1R is detected. In other instances, the expression level of CCR5 and IL3R is detected. In some instances, the expression level of CCR5 and GM-CSFR is detected. In other instances, the expression level of CX3CR1 and IL1R is detected. In some instances, the expression level of CX3CR1 and IL3R is detected. In other instances, the expression level of CX3CR1 and GM-CSFR is detected. In some instances, the expression level of IL1R and IL3R is detected. In other instances, the expression level of IL1R and GM-CSFR is detected. In some instances, the expression level of IL3R and GM-CSFR is detected.

In other instances, the expression level of CR3, CCR5, and CX3CR1 is detected. In some instances, the expression level of CR3, CCR5, and IL1R is detected. In other instances, the expression level of CR3, CCR5, and IL3R is detected. In some instances, the expression level of CR3, CCR5, and GM-CSFR is detected. In other instances, the expression level of CR3, CX3CR1, and IL1R is detected. In some instances, the expression level of CR3, CX3CR1, and IL3R is detected. In other instances, the expression level of CR3, CX3CR1, and GM-CSFR is detected. In some instances, the expression level of CR3, IL1R, and IL3R is detected. In other instances, the expression level of CR3, IL1R, and GM-CSFR is detected. In some instances, the expression level of CR3, IL3R, and GM-CSFR is detected. In other instances, the expression level of CCR5, CX3CR1, and IL1R is detected. In some instances, the expression level of CCR5, CX3CR1, and IL3R is detected. In other instances, the expression level of CCR5, CX3CR1, and GM-CSFR is detected. In some instances, the expression level of CCR5, IL1R, and IL3R is detected. In other instances, the expression level of CCR5, IL1R, and GM-CSFR is detected. In some instances, the expression level of CCR5, IL3R, and GM-CSFR is detected. In other instances, the expression level of CX3CR1, IL1R, and IL3R is detected. In some instances, the expression level of CX3CR1, IL1R, and GM-CSFR is detected. In other instances, the expression level of IL1R, IL3R, and GM-CSFR is detected.

In some instances, the expression level of CR3, CCR5, CX3CR1, and IL1R is detected. In other instances, the expression level of CR3, CCR5, CX3CR1, and IL3R is detected. In some instances, the expression level of CR3, CCR5, CX3CR1, and GM-CSFR is detected. In other instances, the expression level of CR3, CX3CR1, IL1R, and IL3R is detected. In some instances, the expression level of CR3, CX3CR1, IL1R, and GM-CSFR is detected. In other instances, the expression level of CCR5, CX3CR1, IL1R, and IL3R is detected. In some instances, the expression level of CCR5, CX3CR1, IL1R, and GM-CSFR is detected. In other instances, the expression level of CCR5, IL1R, IL3R, GM-CSFR is detected. In some instances, the expression level of CX3CR1, IL1R, IL3R, GM-CSFR is detected.

In other instances, the expression level of CR3, CCR5, CX3CR1, IL1R, and IL3R is detected. In some instances, the expression level of CR3, CCR5, CX3CR1, IL1R, and GM-CSFR is detected. In other instances, the expression level of CCR5, CX3CR1, IL1R, IL3R, GM-CSFR is detected. In some instances, the expression level of CR3, CCR5, CX3CR1, IL1R, IL3R, and GM-CSFR is detected.

In other embodiments, the method further comprises detecting in the biological sample the expression level of one or more additional cytokine receptors such as, e.g., IL1RAPL1, IFNγR, and/or other cytokine receptors known in the art.

In some instances, the expression level of a particular cytokine receptor is the mRNA level of that cytokine receptor. The mRNA level can be detected or measured with an assay such as, e.g., a hybridization assay or an amplification-based assay. In other instances, the expression level of a particular cytokine receptor is the protein level of that cytokine receptor. The protein level can be detected or measured with an assay such as, e.g., an immunoassay (e.g., ELISA), an immunohistochemical assay, or a multiplexed immunoarray.

In some embodiments, the biological sample is a whole blood, serum, plasma, saliva, urine, cerebrospinal fluid, amniotic fluid, nipple aspirate, or tissue sample. In certain instances, the tissue sample is brain tissue. The brain tissue can be from any brain region including the frontal cortex (e.g., dorsolateral prefrontal cortex), anterior cingulate cortex, cerebellar cortex, superior temporal gyms, parietal cortex, nucleus accumbens, amygdala, or combinations thereof.

In certain embodiments, the control expression level is the expression level (e.g., mRNA or protein level) of the one or more cytokine receptors in an individual or a population of individuals without the neuroimmune-based psychiatric disorder, i.e., an age and/or sex-matched control individual or a population of such control individuals. In particular embodiments, the expression level (e.g., mRNA or protein level) of the one or more cytokine receptors detected in the biological sample is decreased by more than 1-fold compared to the control expression level of the one or more cytokine receptors. For example, the expression level (e.g., mRNA or protein level) of the one or more cytokine receptors detected in the biological sample can be decreased by more than about 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.5, 4, 4.5, 5, 5.5, 6, 6.5, 7, 7.5, 8, 8.5, 9, 9.5, or 10-fold, or from about 1 to 2, 1 to 3, 1 to 4, 1 to 5, 1 to 6, 1 to 7, 1 to 8, 1 to 9, 1 to 10, 2 to 3, 2 to 4, or 2 to 5-fold compared to the control expression level of the one or more cytokine receptors. In certain instances, the expression level (e.g., mRNA or protein level) of a specific cytokine receptor is normalized to one or more housekeeping genes and then compared to a normalized expression level (e.g., mRNA or protein level) of the same cytokine receptor in a control individual or a population of control individuals without the neuroimmune-based psychiatric disorder in order to determine the fold difference in the expression level of the cytokine receptor.

In another aspect, the present invention provides a method for the in vivo imaging of brain tissue for determining whether an individual has or is at risk of developing a neuroimmune-based psychiatric disorder, the method comprising:
(a) administering to the individual one or more imaging agents comprising one or more ligands that bind to one or more cytokine receptors selected from the group consisting of CR3, CCR5, CX3CR1, IL1R, IL3R, GM-CSFR, and combinations thereof, wherein a detectable moiety is attached to the one or more ligands; and
(b) detecting the one or more imaging agents in brain tissue of the individual, wherein the individual has or is at risk of developing a neuroimmune-based psychiatric disorder when the level of the one or more imaging agents detected in the frontal cortex is less than the level of the one or more imaging agents detected in an individual or a population of individuals without the neuroimmune-based psychiatric disorder.

In some embodiments, the neuroimmune-based psychiatric disorder is schizophrenia (SZ) or an autism spectrum disorder (ASD). In other embodiments, the neuroimmune-based psychiatric disorder is a mood disorder such as, e.g., major depressive disorder (MDD) or bipolar disorder. In preferred embodiments, the individual is a human.

In certain embodiments, the one or more imaging agents detect any one or a plurality (e.g., at least two, three, four, five, or six) of the cytokine receptors. In particular embodiments, the one or more imaging agents detect one, two, three, or all four cytokine receptors selected from the group consisting of CR3, CCR5, CX3CR1, and IL1R.

In some instances, the one or more imaging agents detect CR3 and CCR5. In other instances, the one or more imaging agents detect CR3 and CX3CR1. In some instances, the one or more imaging agents detect CR3 and IL1R. In other instances, the one or more imaging agents detect CR3 and IL3R. In some instances, the one or more imaging agents detect CR3 and GM-CSFR. In other instances, the one or more imaging agents detect CCR5 and CX3CR1. In some instances, the one or more imaging agents detect CCR5 and IL1R. In other instances, the one or more imaging agents detect CCR5 and IL3R. In some instances, the one or more imaging agents detect CCR5 and GM-CSFR. In other instances, the one or more imaging agents detect CX3CR1 and IL1R. In some instances, the one or more imaging agents detect CX3CR1 and IL3R. In other instances, the one or more imaging agents detect CX3CR1 and GM-CSFR. In some instances, the one or more imaging agents detect IL1R and IL3R. In other instances, the one or more imaging agents detect IL1R and GM-CSFR. In some instances, the one or more imaging agents detect IL3R and GM-CSFR.

In other instances, the one or more imaging agents detect CR3, CCR5, and CX3CR1. In some instances, the one or more imaging agents detect CR3, CCR5, and IL1R. In other instances, the one or more imaging agents detect CR3, CCR5, and IL3R. In some instances, the one or more imaging agents detect CR3, CCR5, and GM-CSFR. In other instances, the one or more imaging agents detect CR3, CX3CR1, and IL1R. In some instances, the one or more imaging agents detect CR3, CX3CR1, and IL3R. In other instances, the one or more imaging agents detect CR3, CX3CR1, and GM-CSFR. In some instances, the one or more imaging agents detect CR3, IL1R, and IL3R. In other instances, the one or more imaging agents detect CR3, IL1R, and GM-CSFR. In some instances, the one or more imaging agents detect CR3, IL3R, and GM-CSFR. In other instances, the one or more imaging agents detect CCR5, CX3CR1, and IL1R. In some instances, the one or more imaging agents detect CCR5, CX3CR1, and IL3R. In other instances, the one or more imaging agents detect CCR5, CX3CR1, and GM-CSFR. In some instances, the one or more imaging agents detect CCR5, IL1R, and IL3R. In other instances, the one or more imaging agents detect CCR5, IL1R, and GM-CSFR. In some instances, the one or more imaging agents detect CCR5, IL3R, and GM-CSFR. In other instances, the one or more imaging agents detect CX3CR1, IL1R, and IL3R. In some instances, the one or more imaging agents detect CX3CR1, IL1R, and GM-CSFR. In other instances, the one or more imaging agents detect IL1R, IL3R, and GM-CSFR.

In some instances, the one or more imaging agents detect CR3, CCR5, CX3CR1, and IL1R. In other instances, the one or more imaging agents detect CR3, CCR5, CX3CR1, and IL3R. In some instances, the one or more imaging agents detect CR3, CCR5, CX3CR1, and GM-CSFR. In other instances, the one or more imaging agents detect CR3, CX3CR1, IL1R, and IL3R. In some instances, the one or more imaging agents detect CR3, CX3CR1, IL1R, and GM-CSFR. In other instances, the one or more imaging agents detect CCR5, CX3CR1, IL1R, and IL3R. In some instances, the one or more imaging agents detect CCR5, CX3CR1, IL1R, and GM-CSFR. In other instances, the one or more imaging agents detect CCR5, IL1R, IL3R, GM-CSFR. In some instances, the one or more imaging agents detect CX3CR1, IL1R, IL3R, GM-CSFR.

In other instances, the one or more imaging agents detect CR3, CCR5, CX3CR1, IL1R, and IL3R. In some instances, the one or more imaging agents detect CR3, CCR5, CX3CR1, IL1R, and GM-CSFR. In other instances, the one or more imaging agents detect CCR5, CX3CR1, IL1R, IL3R, GM-CSFR. In some instances, the one or more imaging agents detect CR3, CCR5, CX3CR1, IL1R, IL3R, and GM-CSFR.

In other embodiments, the method further comprises administering one or more imaging agents comprising one or more ligands that bind to one or more additional cytokine receptors such as, e.g., IL1RAPL1, IFNγR, and/or other cytokine receptors known in the art.

In particular embodiments, the individual or the population of individuals without the neuroimmune-based psychiatric disorder is an age and/or sex-matched control individual or a population of such control individuals. In particular embodiments, the level of the one or more imaging agents detected in the frontal cortex is less (e.g., decreased) by more than 1-fold compared to the level of the one or more imaging agents detected in the same brain region in the control individual or the population of control individuals without the neuroimmune-based psychiatric disorder. For example, the level of the one or more imaging agents detected in the frontal cortex can be less (e.g., decreased) by more than about 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.5, 4, 4.5, 5, 5.5, 6, 6.5, 7, 7.5, 8, 8.5, 9, 9.5, or 10-fold, or from about 1 to 2, 1 to 3, 1 to 4, 1 to 5, 1 to 6, 1 to 7, 1 to 8, 1 to 9, 1 to 10, 2 to 3, 2 to 4, or 2 to 5-fold compared to the control individual or the population of control individuals without the neuroimmune-based psychiatric disorder. In certain instances, the level of a specific imaging agent is normalized to one or more control imaging agents (e.g., directed to housekeeping proteins) and then compared to a normalized level of the same imaging agent in a control individual or a population of control individuals without the neuroimmune-based psychiatric disorder in order to determine the fold difference in the level of the imaging agent.

In some embodiments, the one or more ligands comprises one or more cytokines or receptor-binding fragments thereof, or any other binding reagent such as an antibody that specifically binds to the one or more cytokine receptors. Non-limiting examples of cytokines useful as ligands for the imaging agents of the present invention include IL-1β (e.g., IL1R-binding), RANTES (e.g., CCR5-binding), MIP (e.g., CCR5-binding), CCL3L1 (e.g., CCR5-binding), GM-CSF (e.g., GM-CSFR-binding), IL-3 (e.g., IL3R-binding), CX3CL1 (e.g., CX3CR1-binding), complement component 3b (iC3b) (e.g., CR3-binding), fragments thereof (e.g., receptor-binding fragments thereof), and combinations thereof. Non-limiting examples of antibodies include anti-CR3 antibodies, anti-CCR5 antibodies, anti-CX3CR1 antibodies, anti-IL1R antibodies, anti-IL3R antibodies, anti-GM-CSFR antibodies, and combinations thereof. In particular embodiments, the antibodies specifically bind to the extracellular domain of the one or more cytokine receptors.

In certain embodiments, the detectable moiety attached to the ligand in the imaging agent is a radionuclide. In particular embodiments, the imaging agent is detected by Positron Emission Tomography (PET), Single Photon Emission Computerized Tomography (SPECT), Magnetic Resonance Imaging (MRI), Magnetic Resonance Spectroscopy (MRS), or optical imaging. In other embodiments, the imaging agent is administered intravenously, intracranially, intrathecally, intraspinally, intraperitoneally, intramuscularly, intralesionally, intranasally, orally, or subcutaneously.

In alternative embodiments, step (b) comprises detecting the one or more imaging agents in brain tissue of the individual, wherein the individual has or is at risk of developing a neuroimmune-based psychiatric disorder when the level of the one or more imaging agents detected in the frontal cortex (e.g., DLPFC) is less than the level of the one or more imaging agents detected in a different brain region. Non-limiting examples of different brain regions include the anterior cingulate cortex, cerebellar cortex, superior temporal gyms, parietal cortex, nucleus accumbens, amygdala, and combinations thereof.

In yet another aspect, the present invention provides a method for identifying a compound for treating a neuroimmune-based psychiatric disorder, the method comprising:
(a) contacting the compound with one or more cytokine receptors selected from the group consisting of CR3, CCR5, Cx3CR1, IL1R, IL3R, GM-CSFR, and combinations thereof; and
(b) determining whether the compound increases the expression level or activity of the one or more cytokine receptors, thereby identifying a compound for treating a neuroimmune-based psychiatric disorder.

In some embodiments, the neuroimmune-based psychiatric disorder is schizophrenia (SZ) or an autism spectrum disorder (ASD). In other embodiments, the neuroimmune-based psychiatric disorder is a mood disorder such as, e.g., major depressive disorder (MDD) or bipolar disorder.

In certain embodiments, the compound is contacted with a cell expressing the one or more cytokine receptors. In some instances, the cell expresses any one or a plurality (e.g., at least two, three, four, five, or six) of the cytokine receptors. In particular embodiments, the cell expresses one, two, three, or all four cytokine receptors selected from the group consisting of CR3, CCR5, CX3CR1, and IL1R.

In some instances, the cell expresses CR3 and CCR5. In other instances, the cell expresses CR3 and CX3CR1. In some instances, the cell expresses CR3 and IL1R. In other instances, the cell expresses CR3 and IL3R. In some instances, the cell expresses CR3 and GM-CSFR. In other instances, the cell expresses CCR5 and CX3CR1. In some instances, the cell expresses CCR5 and IL1R. In other instances, the cell expresses CCR5 and IL3R. In some instances, the cell expresses CCR5 and GM-CSFR. In other instances, the cell expresses CX3CR1 and IL1R. In some instances, the cell expresses CX3CR1 and IL3R. In other instances, the cell expresses CX3CR1 and GM-CSFR. In some instances, the cell expresses IL1R and IL3R. In other instances, the cell expresses IL1R and GM-CSFR. In some instances, the cell expresses IL3R and GM-CSFR.

In other instances, the cell expresses CR3, CCR5, and CX3CR1. In some instances, the cell expresses CR3, CCR5, and IL1R. In other instances, the cell expresses CR3, CCR5, and IL3R. In some instances, the cell expresses CR3, CCR5, and GM-CSFR. In other instances, the cell expresses CR3, CX3CR1, and IL1R. In some instances, the cell expresses CR3, CX3CR1, and IL3R. In other instances, the cell expresses CR3, CX3CR1, and GM-CSFR. In some instances, the cell expresses CR3, IL1R, and IL3R. In other instances, the cell expresses CR3, IL1R, and GM-CSFR. In some instances, the cell expresses CR3, IL3R, and GM-CSFR. In other instances, the cell expresses CCR5, CX3CR1, and IL1R. In some instances, the cell expresses CCR5, CX3CR1, and IL3R. In other instances, the cell expresses CCR5, CX3CR1, and GM-CSFR. In some instances, the cell expresses CCR5, IL1R, and IL3R. In other instances, the cell expresses CCR5, IL1R, and GM-CSFR. In some instances, the cell expresses CCR5, IL3R, and GM-CSFR. In other instances, the cell expresses CX3CR1, IL1R, and IL3R. In some instances, the cell expresses CX3CR1, IL1R, and GM-CSFR. In other instances, the cell expresses IL1R, IL3R, and GM-CSFR.

In some instances, the cell expresses CR3, CCR5, CX3CR1, and IL1R. In other instances, the cell expresses CR3, CCR5, CX3CR1, and IL3R. In some instances, the cell expresses CR3, CCR5, CX3CR1, and GM-CSFR. In other instances, the cell expresses CR3, CX3CR1, IL1R, and IL3R. In some instances, the cell expresses CR3, CX3CR1, IL1R, and GM-CSFR. In other instances, the cell expresses CCR5, CX3CR1, IL1R, and IL3R. In some instances, the cell expresses CCR5, CX3CR1, IL1R, and GM-CSFR. In other instances, the cell expresses CCR5, IL1R, IL3R, GM-CSFR. In some instances, the cell expresses CX3CR1, IL1R, IL3R, GM-CSFR.

In other instances, the cell expresses CR3, CCR5, CX3CR1, IL1R, and IL3R. In some instances, the cell expresses CR3, CCR5, CX3CR1, IL1R, and GM-CSFR. In other instances, the cell expresses CCR5, CX3CR1, IL1R, IL3R, GM-CSFR. In some instances, the cell expresses CR3, CCR5, CX3CR1, IL1R, IL3R, and GM-CSFR.

In other embodiments, the method further comprises contacting the compound with one or more additional cytokine receptors such as, e.g., IL1RAPL1, IFNγR, and/or other cytokine receptors known in the art. In related embodiments, the cell further expresses one or more additional cytokine receptors such as, e.g., IL1RAPL1, IFNγR, and/or other cytokine receptors known in the art.

In particular embodiments, the compound increases the expression level or activity of a specific cytokine receptor by more than 1-fold compared to the level of the expression level or activity of the same cytokine receptor not contacted with the compound. For example, the expression level or activity of a specific cytokine receptor contacted with the compound can be increased by more than about 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.5, 4, 4.5, 5, 5.5, 6, 6.5, 7, 7.5, 8, 8.5, 9, 9.5, or 10-fold, or from about 1 to 2, 1 to 3, 1 to 4, 1 to 5, 1 to 6, 1 to 7, 1 to 8, 1 to 9, 1 to 10, 2 to 3, 2 to 4, or 2 to 5-fold compared to the same cytokine receptor not contacted with the compound. In certain instances, the expression level or activity of a specific cytokine receptor contacted with the compound is normalized to one or more housekeeping proteins and then compared to a normalized level of the same cytokine receptor not contacted with the compound in order to determine the fold difference in the level or activity of the cytokine receptor.

In some embodiments, the method further comprises: (c) administering the compound to an animal model selected from the group consisting of a mouse model of maternal immune activation (MIA) and a non-human primate model of MIA. In other embodiments, the method further comprises: (d) determining the effect of the compound on the expression level or activity of the one or more cytokine receptors in the animal model.

In a further aspect, the present invention provides a method for treating a neuroimmune-based psychiatric disorder in an individual in need thereof, the method comprising:
(a) administering to the individual a therapeutically effective amount of a compound identified using the method described herein.

In some embodiments, the neuroimmune-based psychiatric disorder is schizophrenia (SZ) or an autism spectrum disorder (ASD). In other embodiments, the neuroimmune-based psychiatric disorder is a mood disorder such as, e.g., major depressive disorder (MDD) or bipolar disorder. In preferred embodiments, the individual is a human.

In other embodiments, the compound is administered intravenously, intracranially, intrathecally, intraspinally, intraperitoneally, intramuscularly, intralesionally, intranasally, orally, or subcutaneously.

In a related aspect, the present invention provides a method for treating a neuroimmune-based psychiatric disorder in an individual in need thereof, the method comprising:
(a) administering to the individual a therapeutically effective amount of a nucleic acid encoding one or more cytokine receptors selected from the group consisting of CR3, CCR5, Cx3CR1, IL1R, IL3R, GM-CSFR, and combinations thereof.

In some embodiments, the neuroimmune-based psychiatric disorder is schizophrenia (SZ) or an autism spectrum disorder (ASD). In other embodiments, the neuroimmune-based psychiatric disorder is a mood disorder such as, e.g., major depressive disorder (MDD) or bipolar disorder. In preferred embodiments, the individual is a human.

In other embodiments, the nucleic acid is administered intravenously, intracranially, intrathecally, intraspinally, intraperitoneally, intramuscularly, intralesionally, intranasally, orally, or subcutaneously.

In some embodiments, the nucleic acid encodes any one or a plurality (e.g., at least two, three, four, five, or six in tandem as a polycistronic transcript) of the cytokine receptors. In other embodiments, the nucleic acid comprises a plurality (e.g., at least two, three, four, five, or six) of nucleic acids, each encoding a different cytokine receptor (e.g., in individual expression vectors).

In some instances, the nucleic acid encodes CR3 and CCR5. In other instances, the nucleic acid encodes CR3 and CX3CR1. In some instances, the nucleic acid encodes CR3 and IL1R. In other instances, the nucleic acid encodes CR3 and IL3R. In some instances, the nucleic acid encodes CR3 and GM-CSFR. In other instances, the nucleic acid encodes CCR5 and CX3CR1. In some instances, the nucleic acid encodes CCR5 and IL1R. In other instances, the nucleic acid encodes CCR5 and IL3R. In some instances, the nucleic acid encodes CCR5 and GM-CSFR. In other instances, the nucleic acid encodes CX3CR1 and IL1R. In some instances, the nucleic acid encodes CX3CR1 and IL3R. In other instances, the nucleic acid encodes CX3CR1 and GM-CSFR. In some instances, the nucleic acid encodes IL1R and IL3R. In other instances, the nucleic acid encodes IL1R and GM-CSFR. In some instances, the nucleic acid encodes IL3R and GM-CSFR.

In other instances, the nucleic acid encodes CR3, CCR5, and CX3CR1. In some instances, the nucleic acid encodes CR3, CCR5, and IL1R. In other instances, the nucleic acid encodes CR3, CCR5, and IL3R. In some instances, the nucleic acid encodes CR3, CCR5, and GM-CSFR. In other instances, the nucleic acid encodes CR3, CX3CR1, and IL1R. In some instances, the nucleic acid encodes CR3, CX3CR1, and IL3R. In other instances, the nucleic acid encodes CR3, CX3CR1, and GM-CSFR. In some instances, the nucleic acid encodes CR3, IL1R, and IL3R. In other instances, the nucleic acid encodes CR3, IL1R, and GM-CSFR. In some instances, the nucleic acid encodes CR3, IL3R, and GM-CSFR. In other instances, the nucleic acid encodes CCR5, CX3CR1, and IL1R. In some instances, the nucleic acid encodes CCR5, CX3CR1, and IL3R. In other instances, the nucleic acid encodes CCR5, CX3CR1, and GM-CSFR. In some instances, the nucleic acid encodes CCR5, IL1R, and IL3R. In other instances, the nucleic acid encodes CCR5, IL1R, and GM-CSFR. In some instances, the nucleic acid encodes CCR5, IL3R, and GM-CSFR. In other instances, the nucleic acid encodes CX3CR1, IL1R, and IL3R. In some instances, the nucleic acid encodes CX3CR1, IL1R, and GM-CSFR. In other instances, the nucleic acid encodes IL1R, IL3R, and GM-CSFR.

In some instances, the nucleic acid encodes CR3, CCR5, CX3CR1, and IL1R. In other instances, the nucleic acid encodes CR3, CCR5, CX3CR1, and IL3R. In some instances, the nucleic acid encodes CR3, CCR5, CX3CR1, and GM-CSFR. In other instances, the nucleic acid encodes CR3, CX3CR1, IL1R, and IL3R. In some instances, the nucleic acid encodes CR3, CX3CR1, IL1R, and GM-CSFR. In other instances, the nucleic acid encodes CCR5, CX3CR1, IL1R, and IL3R. In some instances, the nucleic acid encodes CCR5, CX3CR1, IL1R, and GM-CSFR. In other instances, the nucleic acid encodes CCR5, IL1R, IL3R, GM-CSFR. In some instances, the nucleic acid encodes CX3CR1, IL1R, IL3R, GM-CSFR.

In other instances, the nucleic acid encodes CR3, CCR5, CX3CR1, IL1R, and IL3R. In some instances, the nucleic acid encodes CR3, CCR5, CX3CR1, IL1R, and GM-CSFR. In other instances, the nucleic acid encodes CCR5, CX3CR1, IL1R, IL3R, GM-CSFR. In some instances, the nucleic acid encodes CR3, CCR5, CX3CR1, IL1R, IL3R, and GM-CSFR.

In some embodiments, the method further comprises administering to the individual a therapeutically effective amount of a nucleic acid encoding one or more additional cytokine receptors such as, e.g., IL1RAPL1, IFNγR, and/or other cytokine receptors known in the art.

In certain embodiments, the nucleic acid is incorporated into a vector such as a bacterial or viral vector. Examples of vectors used for such purposes include expression plasmids capable of directing the expression of the nucleic acid in a target cell. In some instances, the vector is a viral vector system wherein the nucleic acid is incorporated into a viral genome that is capable of transfecting a target cell. In particular embodiments, the nucleic acid can be operably linked to expression and control sequences that can direct expression of the cytokine receptor in the desired target host cells. Thus, the expression of the nucleic acid under appropriate conditions can be achieved in the target cell.

### IV. CYTOKINE RECEPTORS

In certain aspects, the methods of the present invention are useful for determining whether an individual has or is at risk of developing a neuroimmune-based psychiatric disorder by detecting the expression level of one or more cytokine receptors in a biological sample. In certain other aspects, the methods of the present invention are useful for the in vivo imaging of brain tissue by detecting one or more imaging agents such as ligands (e.g., cytokines) that bind to one or more cytokine receptors. In yet other aspects, the methods of the present invention are useful for identifying compounds that modulate (e.g., increase) the expression level or activity of one or more cytokine receptors. The present invention further provides therapeutic methods that target one or more cytokine receptors for the treatment of a neuroimmune-based psychiatric disorder by administering a therapeutic agent described herein.

“Cytokine receptors” are receptors that bind cytokines. The term “cytokine” includes any of a variety of polypeptides or proteins secreted by immune cells that regulate a range of immune system functions and encompasses small cytokines such as chemokines. Cytokine receptors typically belong to one of the following classifications: (1) type I cytokine receptors, whose members have certain conserved motifs in their extracellular amino-acid domain; (2) type II cytokine receptors, whose members are receptors mainly for interferons; (3) immunoglobulin (Ig) superfamily, which are ubiquitously present throughout several cells and tissues of the vertebrate body; (4) tumor necrosis factor receptor family, whose members share a cysteine-rich common extracellular binding domain; (5) chemokine receptors, which are G protein coupled receptors; (5) TGF-beta receptors; and (6) integrin receptors.

In particular embodiments, the cytokine receptors include complement receptor 3 (CR3), C-C chemokine receptor type 5 (CCR5), CX3C chemokine receptor 1 (CX3CR1), interleukin-1 receptor (IL1R), interleukin-3 receptor (IL3R), granulocyte macrophage colony-stimulating factor receptor (GM-CSFR), and combinations thereof. The methods of the present invention may target any one or a plurality (e.g., at least two, three, four, five, or six) of these cytokine receptors. In certain instances, the methods of the present invention target the following combination of four cytokine receptors: CR3, CCR5, Cx3CR1, and IL1R. In other embodiments, the methods of the present invention also target one or more additional cytokine receptors such as interleukin-1 receptor accessory protein-like 1 (IL1RAPL1), interferon gamma receptor 1 (IFNGR1), and/or other cytokine receptors known in the art. Collectively, these cytokine receptors play essential roles in brain development and adaptive function.

Complement receptor 3 (CR3), also known as macrophage-1 antigen or integrin αMβ2 (ITGAM), is a complement receptor consisting of CD11b (integrin αM) and CD18 (integrin β2). It binds to C3b and C4b. CR3 is a human cell surface receptor found on polymorphonuclear leukocytes (mostly neutrophils), NK cells, and mononuclear phagocytes such as macrophages. CR3 is a pattern recognition receptor, capable of recognizing and binding to many molecules found on the surfaces of invading bacteria. CR3 is the receptor for the iC3b fragment of the third complement component and has been implicated in adhesion between monocytes, macrophages, and granulocytes as well as in mediating the uptake of complement-coated particles. Complement has been implicated in tagging weakened, inappropriate synapses for elimination during the period of activity-dependent synaptic pruning in the brain.

C-C chemokine receptor type 5, also known as CCR5 or CD195, is a protein on the surface of white blood cells that is involved in the immune system as it acts as a receptor for chemokines. CCR5 is a G protein-coupled receptor that belongs to the beta chemokine receptor family of integral membrane proteins. CCR5 binds to RANTES, MIP, and CCL3L1. In the brain, CCR5 facilitates microglial activation and migration as well as T-cell entry into the central nervous system (CNS).

CX3C chemokine receptor 1 (CX3CR1), also known as the fractalkine receptor or G-protein coupled receptor 13 (GPR13), is a protein that in humans is encoded by the CX3CR1 gene. The CX3CR1 receptor binds the chemokine CX3CL1 (also called neurotactin or fractalkine), which is expressed in neurons and is thought to mediate the recruitment of CX3CR1-expressing microglia to neurons following injury. In the immune system, fractalkine is a chemokine that mediates adhesion and migration of leukocytes. Fractalkine also plays a role during neural development in regulating the migration of microglia into synapses, which appears to be critical for activity-dependent synapse elimination. CX3CR1 is also a coreceptor for HIV-1, and some variations in this gene lead to increased susceptibility to HIV-1 infection and rapid progression to AIDS.

The interleukin-1 receptor (IL-1R) is a cytokine receptor which binds interleukin-1. Two forms of the receptor exist. The type I receptor is primarily responsible for transmitting the inflammatory effects of interleukin-1 (IL-1), while type II receptors may act as a suppressor of IL-1 activity by competing for IL-1 binding. Also opposing the effects of IL-1 is the IL-1 receptor antagonist (IL-1RA). IL-1R binds the pro-inflammatory cytokine IL-1β and is expressed on both neurons and glia throughout the brain. IL-1R signaling has roles in neurogenesis, neuronal differentiation, plasticity, synapse formation, and neuroexcitotoxicity.

The interleukin-3 receptor (also known as CD123 antigen) is a receptor found on cells which helps transmit the signal of interleukin-3, a soluble cytokine important in the immune system. The receptor belongs to the type I cytokine receptor family and is a heterodimer with a unique alpha chain paired with the common beta (beta c or CD131) subunit. The receptor, found on pluripotent progenitor cells, induces tyrosine phosphorylation within the cell and promotes proliferation and differentiation within the hematopoietic cell lines.

The granulocyte macrophage colony-stimulating factor receptor (GM-CSFR), also known as CD116, binds the hematopoietic growth factor GM-CSF and may be involved in adult neuronal stem cell differentiation. IL3R binds IL-3, which is also known as the multi-CSF (colony-stimulating factor) and has similar action to GM-CSF, namely, promoting HSC (hemotpoetic stem cell) differentiation. The GM-CSFR is normally located on myeloblast, mature neutrophil, but not on any erythroid or megakaryocytic lineage cells. The receptor is a heterodimer composed of at least two different subunits: an α chain and a β chain which is also present in the receptors for IL-3 and IL-5. The α subunit contains a binding site for GM-CSF. The β chain is involved in signal transduction. Association of the α and β subunits results in receptor activation.

Interleukin-1 receptor accessory protein-like 1 (IL1RAPL1) is an IL-1 family member identified in a screen for X-linked intellectual disability and is similar to the interleukin-1 accessory proteins. It is most closely related to interleukin-1 receptor accessory protein-like 2 (IL1RAPL2). Recently, IL1RAPL1 was found to act as a synaptic organizer through its trans-synaptic adhesion with PTPδ.

Interferon gamma receptor 1 (IFNGR1), also known as CD119, is the ligand-binding chain (alpha) of the heterodimeric gamma interferon receptor, which is found on macrophages. IFNGR1 binds interferon gamma.

### V. DETECTION OF GENE EXPRESSION

The detection of the expression level of polynucleotides encoding cytokine receptors or cytokine receptor polypeptides in accordance with the present invention is useful for diagnostic applications, e.g., to determine whether an individual has or is at risk of developing a neuroimmune-based psychiatric disorder. Moreover, the detection of gene expression is useful to identify modulators of the expression level of the cytokine receptor polypeptides or polynucleotides encoding the same.

In certain instances, the presence or level of a particular cytokine receptor is detected at the level of nucleic acid (e.g., mRNA) expression with an assay such as, for example, a hybridization assay or an amplification-based assay. In certain other instances, the presence or level of a particular cytokine receptor is detected at the level of protein expression using, for example, an immunoassay (e.g., ELISA), an immunohistochemical assay, or a multiplexed immunoarray.

A variety of methods of specific DNA and RNA measurement using nucleic acid hybridization techniques are known to those of skill in the art (see, Sambrook, supra). Some methods involve an electrophoretic separation (e.g., Southern blot for detecting DNA, and Northern blot for detecting RNA), but measurement of DNA and RNA can also be carried out in the absence of electrophoretic separation (e.g., by dot blot).

The selection of a nucleic acid hybridization format is not critical. A variety of nucleic acid hybridization formats are known to those skilled in the art. For example, common formats include sandwich assays and competition or displacement assays. Hybridization techniques are generally described in Hames and Higgins Nucleic Acid Hybridization, A Practical Approach, IRL Press (1985); Gall and Pardue, Proc. Natl. Acad. Sci. U.S.A., 63:378-383 (1969); and John et al. Nature, 223:582-587 (1969).

Detection of a hybridization complex may require the binding of a signal-generating complex to a duplex of target and probe polynucleotides or nucleic acids. Typically, such binding occurs through ligand and anti-ligand interactions as between a ligand-conjugated probe and an anti-ligand conjugated with a signal. The binding of the signal generation complex is also readily amenable to accelerations by exposure to ultrasonic energy.

The label may also allow indirect detection of the hybridization complex. For example, where the label is a hapten or antigen, the sample can be detected by using antibodies. In these systems, a signal is generated by attaching fluorescent or enzyme molecules to the antibodies or in some cases, by attachment to a radioactive label (see, e.g., Tijssen, “Practice and Theory of Enzyme Immunoassays,” Laboratory Techniques in Biochemistry and Molecular Biology, Burdon and van Knippenberg Eds., Elsevier (1985), pp. 9-20).

The probes are typically labeled either directly, as with isotopes, chromophores, lumiphores, chromogens, or indirectly, such as with biotin, to which a streptavidin complex may later bind. Thus, the detectable labels can be primary labels (where the label comprises an element that is detected directly or that produces a directly detectable element) or secondary labels (where the detected label binds to a primary label, e.g., as is common in immunological labeling). Typically, labeled signal nucleic acids are used to detect hybridization. Complementary nucleic acids or signal nucleic acids may be labeled by any one of several methods typically used to detect the presence of hybridized polynucleotides. The most common method of detection is the use of autoradiography with ³H, ¹²⁵I, ³⁵S, ¹⁴C, or ³²P-labeled probes or the like.

Other labels include, e.g., ligands that bind to labeled antibodies, fluorophores, chemiluminescent agents, enzymes, and antibodies which can serve as specific binding pair members for a labeled ligand. An introduction to labels, labeling procedures and detection of labels is found in Polak and Van Noorden Introduction to Immunocytochemistry, 2nd ed., Springer Verlag, N.Y. (1997); and in Haugland Handbook of Fluorescent Probes and Research Chemicals, a combined handbook and catalogue Published by Molecular Probes, Inc. (1996).

In general, a detector which monitors a particular probe or probe combination is used to detect the detection reagent label. Typical detectors include spectrophotometers, phototubes and photodiodes, microscopes, scintillation counters, cameras, film and the like, as well as combinations thereof. Examples of suitable detectors are widely available from a variety of commercial sources known to persons of skill in the art. Commonly, an optical image of a substrate comprising bound labeling moieties is digitized for subsequent computer analysis.

Most typically, the amount of RNA is measured by quantifying the amount of label fixed to the solid support by binding of the detection reagent. Typically, the presence of a modulator during incubation will increase or decrease the amount of label fixed to the solid support relative to a control incubation which does not comprise the modulator, or as compared to a baseline established for a particular reaction type. Means of detecting and quantifying labels are well known to those of skill in the art.

In preferred embodiments, the target nucleic acid or the probe is immobilized on a solid support. Solid supports suitable for use in the assays of the invention are known to those of skill in the art. As used herein, a solid support is a matrix of material in a substantially fixed arrangement.

A variety of automated solid-phase assay techniques are also appropriate. For instance, very large scale immobilized polymer arrays (VLSIPS™), available from Affymetrix, Inc. (Santa Clara, Calif.) can be used to detect changes in expression levels of a plurality of genes involved in the same regulatory pathways simultaneously. See, Tijssen, supra., Fodor et al. (1991) Science, 251: 767-777; Sheldon et al. (1993) Clinical Chemistry 39(4): 718-719, and Kozal et al. (1996) Nature Medicine 2(7): 753-759.

Detection can be accomplished, for example, by using a labeled detection moiety that binds specifically to duplex nucleic acids (e.g., an antibody that is specific for RNA-DNA duplexes). One preferred example uses an antibody that recognizes DNA-RNA heteroduplexes in which the antibody is linked to an enzyme (typically by recombinant or covalent chemical bonding). The antibody is detected when the enzyme reacts with its substrate, producing a detectable product. Coutlee et al. (1989) Analytical Biochemistry 181:153-162; Bogulavski (1986) et al. J. Immunol. Methods 89:123-130; Prooijen-Knegt (1982) Exp. Cell Res. 141:397-407; Rudkin (1976) Nature 265:472-473, Stollar (1970) Proc. Nat'l Acad. Sci. USA 65:993-1000; Ballard (1982) Mol. Immunol. 19:793-799; Pisetsky and Caster (1982) Mol. Immunol. 19:645-650; Viscidi et al. (1988) J. Clin. Microbial. 41:199-209; and Kiney et al. (1989) J. Clin. Microbiol. 27:6-12 describe antibodies to RNA duplexes, including homo and heteroduplexes. Kits comprising antibodies specific for DNA:RNA hybrids are available, e.g., from Digene Diagnostics, Inc. (Beltsville, Md.).

In addition to available antibodies, one of skill in the art can easily make antibodies specific for nucleic acid duplexes using existing techniques, or modify those antibodies that are commercially or publicly available. In addition to the art referenced above, general methods for producing polyclonal and monoclonal antibodies are known to those of skill in the art (see, e.g., Paul (3rd ed.) Fundamental Immunology Raven Press, Ltd., NY (1993); Coligan Current Protocols in Immunology Wiley/Greene, NY (1991); Harlow and Lane Antibodies: A Laboratory Manual Cold Spring Harbor Press, NY (1988); Stites et al. (eds.) Basic and Clinical Immunology (4th ed.) Lange Medical Publications, Los Altos, Calif., and references cited therein; Goding Monoclonal Antibodies: Principles and Practice (2d ed.) Academic Press, New York, N.Y., (1986); and Kohler and Milstein Nature 256: 495-497 (1975)). Other suitable techniques for antibody preparation include selection of libraries of recombinant antibodies in phage or similar vectors (see, Huse et al. Science 246:1275-1281 (1989); and Ward et al. Nature 341:544-546 (1989)). Specific monoclonal and polyclonal antibodies and antisera will usually bind with a K_{D} of at least about 0.1 μM, preferably at least about 0.01 μM or better, and most typically and preferably, 0.001 μM or better.

The nucleic acids used in this invention can be either positive or negative probes. Positive probes bind to their targets and the presence of duplex formation is evidence of the presence of the target. Negative probes fail to bind to the suspect target and the absence of duplex formation is evidence of the presence of the target. For example, the use of a wild-type specific nucleic acid probe or PCR primers may serve as a negative probe in an assay sample where only the nucleotide sequence of interest is present.

The sensitivity of the hybridization assays may be enhanced through use of a nucleic acid amplification system that multiplies the target nucleic acid being detected. Examples of such systems include the polymerase chain reaction (PCR) system, in particular RT-PCR or real time PCR, and the ligase chain reaction (LCR) system. Other methods recently described in the art are the nucleic acid sequence based amplification (NASBA, Cangene, Mississauga, Ontario) and Q Beta Replicase systems. These systems can be used to directly identify mutants where the PCR or LCR primers are designed to be extended or ligated only when a selected sequence is present. Alternatively, the selected sequences can be generally amplified using, for example, nonspecific PCR primers and the amplified target region later probed for a specific sequence indicative of a mutation.

An alternative means for determining the level of expression of the nucleic acids of the present invention is in situ hybridization. In situ hybridization assays are well known and are generally described in Angerer et al., Methods Enzymol. 152:649-660 (1987). In an in situ hybridization assay, cells, preferably human cells from a brain region such as the frontal cortex, are fixed to a solid support, typically a glass slide. If DNA is to be probed, the cells are denatured with heat or alkali. The cells are then contacted with a hybridization solution at a moderate temperature to permit annealing of specific probes that are labeled. The probes are preferably labeled with radioisotopes or fluorescent reporters.

Material containing nucleic acid is routinely obtained from individuals. Such material is any biological matter from which nucleic acid can be prepared. As non-limiting examples, material can be whole blood, serum, plasma, saliva, cheek swab, sputum, or other bodily fluid or tissue that contains nucleic acid. In one embodiment, a method of the present invention is practiced with whole blood, which can be obtained readily by non-invasive means and used to prepare total RNA or genomic DNA. In another embodiment, detecting the expression level of cytokine receptors involves amplification of an individual's nucleic acid using the polymerase chain reaction (PCR). Use of PCR for the amplification of nucleic acids is well known in the art (see, e.g., Mullis et al. (Eds.), The Polymerase Chain Reaction, Birkhäuser, Boston, (1994)). In yet another embodiment, PCR amplification is performed using one or more fluorescently labeled primers. In a further embodiment, PCR amplification is performed using one or more labeled or unlabeled primers that contain a DNA minor groove binder.

Any of a variety of different primers can be used to amplify an individual's nucleic acid by PCR in order to detect the expression level of cytokine receptors in a method of the invention. Such primers generally are designed to have sufficient guanine and cytosine content to attain a high melting temperature which allows for a stable annealing step in the amplification reaction. Several computer programs, such as Primer Select, are available to aid in the design of PCR primers.

Applicable PCR amplification techniques are described in, e.g., Ausubel et al., Current Protocols in Molecular Biology, John Wiley & Sons, Inc. New York (1999), Chapter 7 and Supplement 47; Theophilus et al., “PCR Mutation Detection Protocols,” Humana Press, (2002); and Innis et al., PCR Protocols, San Diego, Academic Press, Inc. (1990). General nucleic acid hybridization methods are described in Anderson, “Nucleic Acid Hybridization,” BIOS Scientific Publishers, 1999. Amplification or hybridization of a plurality of transcribed nucleic acid sequences (e.g., mRNA or cDNA) can also be performed from mRNA or cDNA sequences arranged in a microarray. Microarray methods are generally described in Hardiman, “Microarrays Methods and Applications: Nuts & Bolts,” DNA Press, 2003; and Baldi et al., “DNA Microarrays and Gene Expression: From Experiments to Data Analysis and Modeling,” Cambridge University Press, 2002.

A variety of methods of specific protein, polypeptide, and peptide measurement using various antibody-based techniques are known to those of skill in the art (see, Sambrook, supra). As used herein, the term “antibody” includes a population of immunoglobulin molecules, which can be polyclonal or monoclonal and of any isotype, or an immunologically active fragment of an immunoglobulin molecule. Such an immunologically active fragment contains the heavy and light chain variable regions, which make up the portion of the antibody molecule that specifically binds an antigen. For example, an immunologically active fragment of an immunoglobulin molecule known in the art as Fab, Fab′ or F(ab′)₂ is included within the meaning of the term antibody.

A variety of immunoassay techniques, including competitive and non-competitive immunoassays, can be used to detect the presence or level of one or more cytokine receptors in accordance with the methods described herein. The term immunoassay encompasses techniques including, without limitation, enzyme immunoassays (EIA) such as enzyme multiplied immunoassay technique (EMIT), enzyme-linked immunosorbent assay (ELISA), antigen capture ELISA, sandwich ELISA, IgM antibody capture ELISA (MAC ELISA), and microparticle enzyme immunoassay (MEIA); capillary electrophoresis immunoassays (CEIA); radioimmunoassays (RIA); immunoradiometric assays (IRMA); fluorescence polarization immunoassays (FPIA); and chemiluminescence assays (CL). If desired, such immunoassays can be automated. Immunoassays can also be used in conjunction with laser induced fluorescence (see, e.g., Schmalzing and Nashabeh, Electrophoresis, 18:2184-2193 (1997); Bao, J. Chromatogr. B. Biomed. Sci., 699:463-480 (1997)). Liposome immunoassays, such as flow-injection liposome immunoassays and liposome immunosensors, are also suitable for use in the present invention (see, e.g., Rongen et al., J. Immunol. Methods, 204:105-133 (1997)). In addition, nephelometry assays, in which the formation of protein/antibody complexes results in increased light scatter that is converted to a peak rate signal as a function of the marker concentration, are suitable for use in the present invention. Nephelometry assays are commercially available from Beckman Coulter (Brea, Calif.; Kit #449430) and can be performed using a Behring Nephelometer Analyzer (Fink et al., J. Clin. Chem. Clin. Biol. Chem., 27:261-276 (1989)).

Antigen capture ELISA can be useful for detecting the presence or level of one or more cytokine receptors in accordance with the methods described herein. For example, in an antigen capture ELISA, an antibody directed to a cytokine receptor of interest is bound to a solid phase and sample is added such that the cytokine receptor is bound by the antibody. After unbound proteins are removed by washing, the amount of bound receptor can be quantitated using, e.g., a radioimmunoassay (see, e.g., Harlow and Lane, Antibodies: A Laboratory Manual, Cold Spring Harbor Laboratory, New York, 1988)). Sandwich ELISA can also be suitable for use in the present invention. For example, in a two-antibody sandwich assay, a first antibody is bound to a solid support, and the cytokine receptor of interest is allowed to bind to the first antibody. The amount of the cytokine receptor is quantitated by measuring the amount of a second antibody that binds the receptor. The antibodies can be immobilized onto a variety of solid supports, such as magnetic or chromatographic matrix particles, the surface of an assay plate (e.g., microtiter wells), pieces of a solid substrate material or membrane (e.g., plastic, nylon, paper), and the like. An assay strip can be prepared by coating the antibody or a plurality of antibodies in an array on a solid support. This strip can then be dipped into the test sample and processed quickly through washes and detection steps to generate a measurable signal, such as a colored spot.

A radioimmunoassay using, for example, an iodine-125 (¹²⁵I) labeled secondary antibody (Harlow and Lane, supra) is also suitable for use in the present invention. A secondary antibody labeled with a chemiluminescent marker can also be suitable for use in the present invention. A chemiluminescence assay using a chemiluminescent secondary antibody is suitable for sensitive, non-radioactive detection of expression levels. Such secondary antibodies can be obtained commercially from various sources, e.g., Amersham Lifesciences, Inc. (Arlington Heights, Ill.).

Specific immunological binding of an antibody to a cytokine receptor of interest can be detected directly or indirectly. Direct labels include fluorescent or luminescent tags, metals, dyes, radionuclides, and the like, attached to the antibody. An antibody labeled with iodine-125 (¹²⁵I) can be used for determining the levels of one or more cytokine receptors in a sample. A chemiluminescence assay using a chemiluminescent antibody specific for the cytokine receptor is suitable for sensitive, non-radioactive detection of receptor levels. An antibody labeled with fluorochrome is also suitable for determining the levels of one or more cytokine receptors in a sample. Examples of fluorochromes include, without limitation, DAPI, fluorescein, Hoechst 33258, R-phycocyanin, B-phycoerythrin, R-phycoerythrin, rhodamine, Texas red, and lissamine. Secondary antibodies linked to fluorochromes can be obtained commercially, e.g., goat F(ab′)₂ anti-human IgG-FITC is available from Tago Immunologicals (Burlingame, Calif.).

Indirect labels include various enzymes well-known in the art, such as horseradish peroxidase (HRP), alkaline phosphatase (AP), β-galactosidase, urease, and the like. A horseradish-peroxidase detection system can be used, for example, with the chromogenic substrate tetramethylbenzidine (TMB), which yields a soluble product in the presence of hydrogen peroxide that is detectable at 450 nm. An alkaline phosphatase detection system can be used with the chromogenic substrate p-nitrophenyl phosphate, for example, which yields a soluble product readily detectable at 405 nm. Similarly, a β-galactosidase detection system can be used with the chromogenic substrate o-nitrophenyl-β-D-galactopyranoside (ONPG), which yields a soluble product detectable at 410 nm. An urease detection system can be used with a substrate such as urea-bromocresol purple (Sigma Immunochemicals; St. Louis, Mo.). A useful secondary antibody linked to an enzyme can be obtained from a number of commercial sources, e.g., goat F(ab′)₂ anti-human IgG-alkaline phosphatase can be purchased from Jackson ImmunoResearch (West Grove, Pa.).

A signal from the direct or indirect label can be analyzed, for example, using a spectrophotometer to detect color from a chromogenic substrate; a radiation counter to detect radiation such as a gamma counter for detection of ¹²⁵I; or a fluorometer to detect fluorescence in the presence of light of a certain wavelength. For detection of enzyme-linked antibodies, a quantitative analysis of the amount of cytokine receptor levels can be made using a spectrophotometer such as an EMAX Microplate Reader (Molecular Devices; Menlo Park, Calif.) in accordance with the manufacturer's instructions. If desired, the assays described herein can be automated or performed robotically, and the signal from multiple samples can be detected simultaneously.

Flow cytometry can be used to detect the presence or level of one or more cytokine receptors. Such flow cytometric assays include bead-based immunoassays (see, e.g., Bishop and Davis, J. Immunol. Methods, 210:79-87 (1997); McHugh et al., J. Immunol. Methods, 116:213 (1989); Scillian et al., Blood, 73:2041 (1989)).

Phage display technology for expressing a recombinant antigen specific for a cytokine receptor can also be used. Phage particles expressing an antigen specific for a cytokine receptor can be anchored, if desired, to a multi-well plate using an antibody such as an anti-phage monoclonal antibody (Felici et al., “Phage-Displayed Peptides as Tools for Characterization of Human Sera” in Abelson (Ed.), Methods in Enzymol., 267, San Diego: Academic Press, Inc. (1996)).

Quantitative Western blotting can also be used to detect or determine the presence or level of one or more cytokine receptors in a sample. Western blots can be quantitated by well-known methods such as scanning densitometry or phosphorimaging. As a non-limiting example, protein samples are electrophoresed on 10% SDS-PAGE Laemmli gels. Primary murine monoclonal antibodies are reacted with the blot, and antibody binding can be confirmed to be linear using a preliminary slot blot experiment. Goat anti-mouse horseradish peroxidase-coupled antibodies (BioRad) are used as the secondary antibody, and signal detection performed using chemiluminescence, for example, with the Renaissance chemiluminescence kit (New England Nuclear; Boston, Mass.) according to the manufacturer's instructions. Autoradiographs of the blots are analyzed using a scanning densitometer (Molecular Dynamics; Sunnyvale, Calif.) and normalized to a positive control. Values are reported, for example, as a ratio between the actual value to the positive control (densitometric index). Such methods are well known in the art as described, for example, in Parra et al., J. Vasc. Surg., 28:669-675 (1998).

Alternatively, a variety of immunohistochemical assay techniques can be used to detect or determine the presence or level of one or more cytokine receptors in a sample. The term “immunohistochemical assay” encompasses techniques that utilize the visual detection of fluorescent dyes or enzymes coupled (i.e., conjugated) to antibodies that react with the cytokine receptor of interest using fluorescent microscopy or light microscopy and includes, without limitation, direct fluorescent antibody assay, indirect fluorescent antibody (IFA) assay, anticomplement immunofluorescence, avidin-biotin immunofluorescence, and immunoperoxidase assays.

Alternatively, the presence or level of a cytokine receptor of interest can be determined by detecting or quantifying the amount of the purified receptor. Purification of the cytokine receptor can be achieved, for example, by high pressure liquid chromatography (HPLC), alone or in combination with mass spectrometry (e.g., MALDI/MS, MALDI-TOF/MS, SELDI-TOF/MS, tandem MS, etc.). Qualitative or quantitative detection of a cytokine receptor of interest can also be determined by well-known methods including, without limitation, Bradford assays, Coomassie blue staining, silver staining, assays for radiolabeled protein, and mass spectrometry.

The analysis of a plurality of cytokine receptors may be carried out separately or simultaneously with one test sample. For separate or sequential assay of cytokine receptors, suitable apparatuses include clinical laboratory analyzers such as the ElecSys (Roche), the AxSym (Abbott), the Access (Beckman), the ADVIA®, the CENTAUR® (Bayer), and the NICHOLS ADVANTAGE® (Nichols Institute) immunoassay systems. Preferred apparatuses or protein chips perform simultaneous assays of a plurality of cytokine receptors on a single surface. Particularly useful physical formats comprise surfaces having a plurality of discrete, addressable locations for the detection of a plurality of different cytokine receptors. Such formats include protein microarrays, or “protein chips” (see, e.g., Ng et al., J. Cell Mol. Med., 6:329-340 (2002)) and certain capillary devices (see, e.g., U.S. Pat. No. 6,019,944). In these embodiments, each discrete surface location may comprise antibodies to immobilize one or more cytokine receptors for detection at each location. Surfaces may alternatively comprise one or more discrete particles (e.g., microparticles or nanoparticles) immobilized at discrete locations of a surface, where the microparticles comprise antibodies to immobilize one or more cytokine receptors for detection.

### VI. IMAGING APPLICATIONS

In certain other aspects, the methods of the present invention are useful for the in vivo imaging of brain tissue (e.g., a brain region of interest such as the frontal cortex) by detecting one or more optical imaging agents (e.g., radiotracers or imaging probes) that bind to one or more cytokine receptors. In particular embodiments, the imaging agent comprises a ligand for a cytokine receptor (e.g., a cytokine, fragment thereof, or other binding reagent such as an antibody that specifically binds the cytokine receptor) and a detectable moiety attached thereto.

The imaging agents can be administered either systemically or locally to a brain region to be imaged, prior to the imaging procedure. Generally, the imaging agents are administered in doses effective to achieve the desired optical image of brain tissue. Such doses may vary widely, depending upon the particular imaging agent employed, the brain region subjected to the imaging procedure, the imaging equipment being used, and the like.

In some embodiments, the imaging agents described herein are used to directly stain or label a sample so that the sample can be identified or quantitated. For instance, a specific imaging agent can be added as part of an assay for a biological target analyte (e.g., antigen), as a detectable tracer element in a biological or non-biological fluid, or for other in vitro purposes known to one of skill in the art. Typically, the sample is obtained directly from a liquid source or as a wash from a solid material (organic or inorganic) or a growth medium in which cells have been introduced for culturing, or a buffer solution in which cells have been placed for evaluation. Where the sample comprises cells, the cells are optionally single cells, including microorganisms, or multiple cells associated with other cells in two or three dimensional layers, including multicellular organisms, embryos, tissues, biopsies, filaments, biofilms, and the like.

A detectable response generally refers to a change in, or occurrence of, an optical signal that is detectable either by observation or instrumentally. In certain instances, the detectable response is radioactivity (i.e., radiation), including alpha particles, beta particles, nucleons, electrons, positrons, neutrinos, and gamma rays emitted by a radioactive substance such as a radionuclide. In certain other instances, the detectable response is fluorescence or a change in fluorescence, e.g., a change in fluorescence intensity, fluorescence excitation or emission wavelength distribution, fluorescence lifetime, and/or fluorescence polarization. One of skill in the art will appreciate that the degree and/or location of labeling in an individual or sample can be compared to a standard or control (e.g., healthy brain tissue).

When used in imaging applications, the imaging agents described herein typically have a detectable moiety covalently or noncovalently attached to the ligand. Suitable detectable moieties include, but are not limited to, radionuclides, detectable tags, fluorophores, fluorescent proteins, enzymatic proteins, and the like. One of skill in the art will be familiar with methods for attaching detectable moieties to functional groups present on the ligand. For example, the detectable moiety can be directly attached to the ligand via covalent attachment of the detectable moiety to a primary amine group present in the ligand. One of skill in the art will appreciate that a detectable moiety can also be bound to the ligand via noncovalent interactions (e.g., ionic bonds, hydrophobic interactions, hydrogen bonds, Van der Waals forces, dipole-dipole bonds, etc.).

In certain instances, the ligand is radiolabeled with a radionuclide by directly attaching the radionuclide to the ligand. In certain other instances, a benzoyl group labeled with the radionuclide is directly attached to the ligand. For example, 4-[¹⁸F]-fluorobenzoic acid (“[¹⁸F]FBA”) or 4-[¹⁹F]-fluorobenzoic acid (“[¹⁹F]FBA”) can be used to radiolabel the ligand. In further instances, the radionuclide is bound to a chelating agent or chelating agent-linker attached to the ligand. Suitable radionuclides for direct conjugation include, without limitation, ¹⁸F, ¹⁹F, ¹²⁴I, ¹²⁵I, ¹³¹I, and mixtures thereof. Suitable radionuclides for use with a chelating agent include, without limitation, ⁴⁷Sc, ⁶⁴Cu, ⁶⁷Cu, ⁸⁹Sr, ⁸⁶Y, ⁸⁷Y, ⁹⁰Y, ¹⁰⁵Rh, ¹¹¹Ag, ¹¹¹In, ^{117m}Sn, ¹⁴⁹Pm, ¹⁵³Sm, ¹⁶⁶Ho, ¹⁷⁷Lu, ¹⁸⁶Re, ¹⁸⁸Re, ²¹¹At, ²¹²Bi, and mixtures thereof. Suitable chelating agents include, but are not limited to, DOTA, NOTA, NOTA-TCO, BAD, TETA, DTPA, EDTA, NTA, HDTA, their phosphonate analogs, and mixtures thereof. One of skill in the art will be familiar with methods for attaching radionuclides, chelating agents, and chelating agent-linkers to the ligand. In particular, attachment can be conveniently accomplished using, for example, commercially available bifunctional linking groups (generally heterobifunctional linking groups) that can be attached to a functional group present in a non-interfering position on the ligand and then further linked to a radionuclide, chelating agent, or chelating agent-linker.

Non-limiting examples of fluorophores or fluorescent dyes suitable for use as detectable moieties include Alexa Fluor® dyes (Invitrogen Corp.; Carlsbad, Calif.), fluorescein, fluorescein isothiocyanate (FITC), Oregon Green™; rhodamine, Texas red, tetrarhodamine isothiocynate (TRITC), CyDye™ fluors (e.g., Cy2, Cy3, Cy5), and the like.

Examples of fluorescent proteins suitable for use as detectable moieties include, but are not limited to, green fluorescent protein, red fluorescent protein (e.g., DsRed), yellow fluorescent protein, cyan fluorescent protein, blue fluorescent protein, and variants thereof (see, e.g., U.S. Pat. Nos. 6,403,374, 6,800,733, and 7,157,566). Specific examples of GFP variants include, but are not limited to, enhanced GFP (EGFP), destabilized EGFP, the GFP variants described in Doan et al., Mol. Microbiol., 55:1767-1781 (2005), the GFP variant described in Crameri et al., Nat. Biotechnol., 14:315-319 (1996), the cerulean fluorescent proteins described in Rizzo et al., Nat. Biotechnol., 22:445 (2004) and Tsien, Annu. Rev. Biochem., 67:509 (1998), and the yellow fluorescent protein described in Nagal et al., Nat. Biotechnol., 20:87-90 (2002). DsRed variants are described in, e.g., Shaner et al., Nat. Biotechnol., 22:1567-1572 (2004), and include mStrawberry, mCherry, mOrange, mBanana, mHoneydew, and mTangerine. Additional DsRed variants are described in, e.g., Wang et al., Proc. Natl. Acad. Sci. U.S.A., 101:16745-16749 (2004) and include mRaspberry and mPlum. Further examples of DsRed variants include mRFPmars described in Fischer et al., FEBS Lett., 577:227-232 (2004) and mRFPruby described in Fischer et al., FEBS Lett., 580:2495-2502 (2006).

In other embodiments, the detectable moiety that is bound to a ligand comprises a detectable tag such as, for example, biotin, avidin, streptavidin, or neutravidin. In further embodiments, the detectable moiety comprises an enzymatic protein including, but not limited to, luciferase, chloramphenicol acetyltransferase, β-galactosidase, β-glucuronidase, horseradish peroxidase, xylanase, alkaline phosphatase, and the like.

Any device or method known in the art for detecting the radioactive emissions of radionuclides in an individual is suitable for use in the present invention. For example, methods such as Single Photon Emission Computerized Tomography (SPECT), which detects the radiation from a single photon gamma-emitting radionuclide using a rotating gamma camera, and radionuclide scintigraphy, which obtains an image or series of sequential images of the distribution of a radionuclide in tissues, organs, or body systems using a scintillation gamma camera, may be used for detecting the radiation emitted from a radiolabeled ligand. Positron emission tomography (PET) is another suitable technique for detecting radiation in an individual. Miniature and flexible radiation detectors intended for medical use are produced by Intra-Medical LLC (Santa Monica, Calif.). Magnetic Resonance Imaging (MRI) or any other imaging technique known to one of skill in the art is also suitable for detecting the radioactive emissions of radionuclides. Regardless of the method or device used, such detection is aimed at determining the level of an imaging agent in a brain region of interest (e.g., the frontal cortex), with such level being an indicator of whether an individual has or is at risk of developing a neuroimmune-based psychiatric disorder, e.g., compared to the level of the imaging agent in the same brain region of an individual or a population of individuals without the neuroimmune-based psychiatric disorder.

Non-invasive fluorescence imaging of animals and humans can also provide in vivo diagnostic or prognostic information and can be used in a wide variety of clinical specialties. For instance, techniques have been developed over the years for simple ocular observations following UV excitation to sophisticated spectroscopic imaging using advanced equipment (see, e.g., Andersson-Engels et al., Phys. Med. Biol., 42:815-824 (1997)). Specific devices or methods known in the art for the in vivo detection of fluorescence, e.g., from fluorophores or fluorescent proteins, include, but are not limited to, in vivo near-infrared fluorescence (see, e.g., Frangioni, Curr. Opin. Chem. Biol., 7:626-634 (2003)), the Maestro™ in vivo fluorescence imaging system (Cambridge Research & Instrumentation, Inc.; Woburn, Mass.), in vivo fluorescence imaging using a flying-spot scanner (see, e.g., Ramanujam et al., IEEE Transactions on Biomedical Engineering, 48:1034-1041 (2001)), and the like.

Other methods or devices for detecting an optical response include, without limitation, visual inspection, CCD cameras, video cameras, photographic film, laser-scanning devices, fluorometers, photodiodes, quantum counters, epifluorescence microscopes, scanning microscopes, flow cytometers, fluorescence microplate readers, and signal amplification using photomultiplier tubes.

### VII. SCREENING FOR MODULATORS OF CYTOKINE RECEPTOR EXPRESSION

A number of different screening protocols can be utilized to identify compounds that modulate the level of expression or activity of one or more cytokine receptors of interest in cells, particularly mammalian cells, and especially human cells. In general terms, the screening methods involve screening a plurality of compounds to identify a compound that modulates the expression level or activity of a cytokine receptor of interest by binding to the cytokine receptor, by activating expression of the cytokine receptor, by modulating the binding of another molecule (e.g., a ligand such as a cytokine) to the cytokine receptor, and the like.

A. Binding Assays

Preliminary screens can be conducted by screening for compounds capable of binding to a cytokine receptor of interest, as at least some of the compounds so identified are likely modulators of cytokine receptor expression and/or activity. The binding assays usually involve contacting a cytokine receptor of interest with one or more test compounds and allowing sufficient time for the cytokine receptor and test compounds to form a binding complex. Any binding complexes formed can be detected using any of a number of established analytical techniques. Protein binding assays include, but are not limited to, methods that measure co-precipitation, co-migration on non-denaturing SDS-polyacrylamide gels, and co-migration on Western blots. The cytokine receptor utilized in such assays can be naturally expressed, cloned, or synthesized. Binding assays are also useful, e.g., for identifying antibodies, co-receptors, or other molecules that bind a cytokine receptor of interest.

B. Expression Assays

Certain screening methods involve screening for a compound that up or down-regulates the expression (e.g., mRNA or protein level) of a cytokine receptor of the invention. Such methods generally involve conducting cell-based assays in which test compounds are contacted with one or more cells expressing a cytokine receptor of interest and then detecting an increase or decrease in expression (either transcript, translation product, or catalytic product). Some assays are performed with peripheral cells, or other cells, that express an endogenous cytokine receptor of interest.

Polypeptide or polynucleotide expression can be detected in a number of different ways. As a non-limiting example, the expression level of a polynucleotide can be determined by probing the mRNA expressed in a cell with a probe that specifically hybridizes with a transcript (or complementary nucleic acid derived therefrom) of a cytokine receptor of interest. Probing can be conducted by lysing the cells and conducting Northern blots or without lysing the cells using in situ hybridization techniques. Alternatively, the expression level of a polypeptide can be detected using immunological methods in which a cell lysate is probed with antibodies that specifically bind to a cytokine receptor of interest.

Other cell-based assays are reporter assays conducted with cells that do not express a cytokine receptor of interest. Certain of these assays are conducted with a heterologous nucleic acid construct that includes a promoter of a polynucleotide encoding a cytokine receptor of interest that is operably linked to a reporter gene that encodes a detectable product. A number of different reporter genes can be utilized. Some reporters are inherently detectable. An example of such a reporter is green fluorescent protein that emits fluorescence that can be detected with a fluorescence detector. Other reporters generate a detectable product. Often such reporters are enzymes. Exemplary enzyme reporters include, but are not limited to, β-glucuronidase, chloramphenicol acetyl transferase (CAT); Alton and Vapnek (1979) Nature 282:864-869), luciferase, β-galactosidase, green fluorescent protein (GFP) and alkaline phosphatase (Toh et al. (1980) Eur. J. Biochem. 182:231-238; and Hall et al. (1983) J. Mol. Appl. Gen. 2:101).

In these assays, cells harboring the reporter construct are contacted with a test compound. A test compound that either activates the promoter by binding to it or triggers a cascade that produces a molecule that activates the promoter causes expression of the detectable reporter. Certain other reporter assays are conducted with cells that harbor a heterologous construct that includes a transcriptional control element that activates expression of a polynucleotide encoding a cytokine receptor of interest and a reporter operably linked thereto. Here, too, a compound that binds to the transcriptional control element to activate expression of the reporter or that triggers the formation of a compound that binds to the transcriptional control element to activate reporter expression, can be identified by the generation of signal associated with reporter expression.

The level of expression or activity of a cytokine receptor of interest can be compared to a baseline value. As indicated above, the baseline value can be a value for a control sample or a statistical value that is representative of expression levels for a control population (e.g., healthy individuals not having or at risk for neuroimmune-based psychiatric disorders). Expression levels can also be determined for cells that do not express a cytokine receptor of interest as a negative control. Such cells generally are otherwise substantially genetically the same as the test cells.

A variety of different types of cells can be utilized in the reporter assays. Cells that express an endogenous cytokine receptor of interest include, e.g., brain cells, including cells from the frontal cortex (e.g., dorsolateral prefrontal cortex), cerebellum, anterior cingulate cortex, amygdala, hippocampus, or nucleus accumbens. Cells that do not endogenously express a cytokine receptor of interest can be prokaryotic, but are preferably eukaryotic. The eukaryotic cells can be any of the cells typically utilized in generating cells that harbor recombinant nucleic acid constructs. Exemplary eukaryotic cells include, but are not limited to, yeast, and various higher eukaryotic cells such as the COS, CHO and HeLa cell lines.

Various controls can be conducted to ensure that an observed activity is authentic including running parallel reactions with cells that lack the reporter construct or by not contacting a cell harboring the reporter construct with test compound. Compounds can also be further validated as described below.

C. Validation

Compounds that are initially identified by any of the foregoing screening methods can be further tested to validate the apparent activity. Preferably, such studies are conducted with suitable animal models. The basic format of such methods involves administering a lead compound identified during an initial screen to an animal that serves as a model for humans and then determining if the expression or activity of a cytokine receptor of interest is in fact modulated (e.g., increased or decreased). The animal models utilized in validation studies generally are mammals of any kind. Specific examples of suitable animals include, but are not limited to, primates (e.g., monkeys), mice, and rats.

In particular embodiments, the animal model is a maternal immune activation (MIA) mouse or non-human primate (NHP) model. The MIA model is one of the most commonly used animal models of neuroimmune-based psychiatric disorders such as schizophrenia and autism spectrum disorder with reproducible serological, neuropathological, and behavioral phenotypes. The two main inflammatory agents used to develop this model are poly I:C, which mimics a viral infection, and LPS, which mimics a bacterial infection. When injected systemically, both of these agents induce an innate inflammatory response consisting primarily of overt cytokine induction both peripherally and centrally. Exemplary protocols for the MIA mouse and NHP models are described in the Example below.

D. Exemplary Modulators

The compounds tested as modulators of the one or more cytokine receptors of interest can be any small chemical compound or a biological entity such as a protein, polypeptide, peptide, antibody or antigen-binding fragment thereof, sugar, polysaccharide, oligosaccharide, polynucleotide, oligonucleotide, or lipid. Typically, test compounds will be small chemical molecules and proteins or polypeptides. Essentially any chemical compound can be used as a potential modulator or ligand in the assays of the invention, although most often compounds that can be dissolved in aqueous or organic (especially DMSO-based) solutions are used. The assays are designed to screen large chemical libraries by automating the assay steps and providing compounds from any convenient source to assays, which are typically run in parallel (e.g., in microtiter formats on microtiter plates in robotic assays). It will be appreciated that there are many suppliers of chemical compounds, including Sigma (St. Louis, Mo.), Aldrich (St. Louis, Mo.), Sigma-Aldrich (St. Louis, Mo.), Fluka Chemika-Biochemica Analytika (Buchs, Switzerland) and the like. Modulators also include agents designed to reduce the level of mRNA of a cytokine receptor of interest (e.g. siRNA molecules, antisense molecules, ribozymes, DNAzymes, and the like) or the level of translation from an mRNA.

In certain embodiments, high throughput screening methods involve providing a combinatorial chemical or peptide library containing a large number of potential therapeutic compounds (potential modulator or ligand compounds). Such “combinatorial chemical libraries” or “ligand libraries” are then screened in one or more assays, as described herein, to identify those library members (particular chemical species or subclasses) that display a desired characteristic activity. The compounds thus identified can serve as conventional “lead compounds” or can themselves be used as potential or actual therapeutics.

A combinatorial chemical library is a collection of diverse chemical compounds generated by either chemical synthesis or biological synthesis, by combining a number of chemical “building blocks” such as reagents. For example, a linear combinatorial chemical library such as a polypeptide library is formed by combining a set of chemical building blocks (amino acids) in every possible way for a given compound length (i.e., the number of amino acids in a polypeptide compound). Millions of chemical compounds can be synthesized through such combinatorial mixing of chemical building blocks.

Preparation and screening of combinatorial chemical libraries is well known to those of skill in the art. Such combinatorial chemical libraries include, but are not limited to, peptide libraries (see, e.g., U.S. Pat. No. 5,010,175, Furka, Int. J. Pept. Prot. Res. 37:487-493 (1991) and Houghton et al., Nature 354:84-88 (1991)). Other chemistries for generating chemical diversity libraries can also be used. Such chemistries include, but are not limited to, peptoids (e.g., PCT Publication No. WO 91/19735), encoded peptides (e.g., PCT Publication WO 93/20242), random bio-oligomers (e.g., PCT Publication No. WO 92/00091), benzodiazepines (e.g., U.S. Pat. No. 5,288,514), diversomers such as hydantoins, benzodiazepines and dipeptides (Hobbs et al., Proc. Nat. Acad. Sci. USA 90:6909-6913 (1993)), vinylogous polypeptides (Hagihara et al., J. Amer. Chem. Soc. 114:6568 (1992)), nonpeptidal peptidomimetics with glucose scaffolding (Hirschmann et al., J. Amer. Chem. Soc. 114:9217-9218 (1992)), analogous organic syntheses of small compound libraries (Chen et al., J. Amer. Chem. Soc. 116:2661 (1994)), oligocarbamates (Cho et al., Science 261:1303 (1993)), peptidyl phosphonates (Campbell et al., J. Org. Chem. 59:658 (1994)), nucleic acid libraries (see Ausubel, Berger and Sambrook, all supra), peptide nucleic acid libraries (see, e.g., U.S. Pat. No. 5,539,083), antibody libraries (see, e.g., Vaughn et al., Nature Biotechnology, 14(3):309-314 (1996) and PCT/US96/10287), carbohydrate libraries (see, e.g., Liang et al., Science, 274:1520-1522 (1996) and U.S. Pat. No. 5,593,853), and small organic molecule libraries.

Devices for the preparation of combinatorial libraries are commercially available (see, e.g., 357 MPS, 390 MPS, Advanced Chem Tech, Louisville Ky.; Symphony, Rainin, Woburn, Mass.; 433A Applied Biosystems, Foster City, Calif.; 9050 Plus, Millipore, Bedford, Mass.). In addition, numerous combinatorial libraries are themselves commercially available (see, e.g., ComGenex, Princeton, N.J.; Tripos, Inc., St. Louis, Mo.; 3D Pharmaceuticals, Exton, Pa.; Martek Biosciences, Columbia, Md., etc.).

E. Solid State and Soluble High Throughput Assays

In the high throughput assays of the invention, it is possible to screen up to several thousand different modulators or ligands in a single day. In particular, each well of a microtiter plate can be used to run a separate assay against a selected potential modulator, or, if concentration or incubation time effects are to be observed, every 5-10 wells can test a single modulator. Thus, a single standard microtiter plate can assay about 100 (e.g., 96) modulators. If 1536 well plates are used, then a single plate can easily assay from about 100 to about 1500 different compounds. It is possible to assay several different plates per day; assay screens for up to about 6,000-20,000 different compounds are possible using the integrated systems of the invention. More recently, microfluidic approaches to reagent manipulation have been developed.

The molecule of interest can be bound to the solid state component directly or indirectly via covalent or non-covalent linkage, e.g., via a tag. The tag can be any of a variety of components. In general, a molecule that binds the tag (a tag binder) is fixed to a solid support, and the tagged molecule of interest is attached to the solid support by interaction of the tag and the tag binder.

A number of tags and tag binders can be used, based upon known molecular interactions well described in the literature. For example, where a tag has a natural binder, for example, biotin, protein A, or protein G, it can be used in conjunction with appropriate tag binders (e.g., avidin, streptavidin, neutravidin, the Fc region of an immunoglobulin, etc.). Antibodies to molecules with natural binders such as biotin are also widely available and appropriate tag binders (see, SIGMA Immunochemicals 1998 catalogue SIGMA, St. Louis Mo.).

Similarly, any haptenic or antigenic compound can be used in combination with an appropriate antibody to form a tag/tag binder pair. Thousands of specific antibodies are commercially available and many additional antibodies are described in the literature. For example, in one common configuration, the tag is a first antibody and the tag binder is a second antibody which recognizes the first antibody. In addition to antibody-antigen interactions, receptor-ligand interactions are also appropriate as tag and tag-binder pairs, such as agonists and antagonists of cell membrane receptors (e.g., cell receptor-ligand interactions such as transferrin, c-kit, viral receptor ligands, cytokine receptors, immunoglobulin receptors, the cadherin family, the integrin family, the selectin family, and the like; see, e.g., Pigott & Power, The Adhesion Molecule Facts Book I (1993)). Similarly, toxins and venoms, viral epitopes, hormones (e.g., opiates, steroids, etc.), intracellular receptors (e.g., which mediate the effects of various small ligands, including steroids, thyroid hormone, retinoids and vitamin D; peptides), drugs, lectins, sugars, nucleic acids (both linear and cyclic polymer configurations), oligosaccharides, proteins, phospholipids, and antibodies can all interact with various cell receptors.

Synthetic polymers, such as polyurethanes, polyesters, polycarbonates, polyureas, polyamides, polyethyleneimines, polyarylene sulfides, polysiloxanes, polyimides, and polyacetates can also form an appropriate tag or tag binder. Many other tag/tag binder pairs are also useful in assay systems described herein, as would be apparent to one of skill upon review of this disclosure.

Common linkers such as peptides, polyethers, and the like can also serve as tags, and include polypeptide sequences, such as poly-Gly sequences of between about 5 and 200 amino acids. Such flexible linkers are known to those of skill in the art. For example, poly(ethylene glycol) linkers are available from Shearwater Polymers, Inc., Huntsville, Ala. These linkers optionally have amide linkages, sulfhydryl linkages, or heterofunctional linkages.

Tag binders are fixed to solid substrates using any of a variety of methods currently available. Solid substrates are commonly derivatized or functionalized by exposing all or a portion of the substrate to a chemical reagent which fixes a chemical group to the surface which is reactive with a portion of the tag binder. For example, groups which are suitable for attachment to a longer chain portion would include amines, hydroxyl, thiol, and carboxyl groups. Aminoalkylsilanes and hydroxyalkylsilanes can be used to functionalize a variety of surfaces, such as glass surfaces. The construction of such solid phase biopolymer arrays is well described in the literature (see, e.g., Merrifield, J. Am. Chem. Soc. 85:2149-2154 (1963) (describing solid phase synthesis of, e.g., peptides); Geysen et al., J. Immun. Meth. 102:259-274 (1987) (describing synthesis of solid phase components on pins); Frank and Doring, Tetrahedron 44:60316040 (1988) (describing synthesis of various peptide sequences on cellulose disks); Fodor et al., Science, 251:767-777 (1991); Sheldon et al., Clinical Chemistry 39(4):718-719 (1993); and Kozal et al., Nature Medicine 2(7):753759 (1996) (all describing arrays of biopolymers fixed to solid substrates). Non-chemical approaches for fixing tag binders to substrates include other common methods, such as heat, cross-linking by UV radiation, and the like.

In certain aspects, the present invention provides in vitro assays for identifying, in a high throughput format, compounds that can modulate the expression or activity of a cytokine receptor of interest. In some embodiments, the methods of the present invention include a control reaction. For each of the assay formats described, “no modulator” control reactions that do not include a modulator provide a background level of binding activity.

In some assays, it will be desirable to have positive controls to ensure that the components of the assays are working properly. At least two types of positive controls are appropriate. First, a known activator of a cytokine receptor of interest can be incubated with one sample of the assay, and the resulting increase in signal resulting from an increased expression level or activity of the cytokine receptor of interest determined according to the methods described herein. Second, a known inhibitor of a cytokine receptor of interest can be added, and the resulting decrease in signal for the expression or activity can be similarly detected.

F. Computer-Based Assays

Yet another assay for compounds that modulate the expression or activity of a cytokine receptor of interest involves computer assisted drug design, in which a computer system is used to generate a three-dimensional structure of the cytokine receptor of interest based on the structural information encoded by its amino acid or nucleotide sequence. The input sequence interacts directly and actively with a pre-established algorithm in a computer program to yield secondary, tertiary, and quaternary structural models of the molecule. Similar analyses can be performed on potential ligands or binding partners of the cytokine receptor of interest. The models of the protein or nucleotide structure are then examined to identify regions of the structure that have the ability to bind, e.g., a cytokine receptor of interest. These regions are then used to identify polypeptides that bind to a cytokine receptor of interest.

The three-dimensional structural model of a protein is generated by entering protein amino acid sequences of at least 10 amino acid residues or corresponding nucleic acid sequences encoding a potential receptor into the computer system. The amino acid sequences encoded by the nucleic acid sequences represent the primary sequences or subsequences of the proteins, which encode the structural information of the proteins. At least 10 residues of an amino acid sequence (or a nucleotide sequence encoding 10 amino acids) are entered into the computer system from computer keyboards, computer readable substrates that include, but are not limited to, electronic storage media (e.g., magnetic diskettes, tapes, cartridges, and chips), optical media (e.g., CD ROM), information distributed by internet sites, and by RAM. The three-dimensional structural model of the protein is then generated by the interaction of the amino acid sequence and the computer system, using software known to those of skill in the art.

The amino acid sequence represents a primary structure that encodes the information necessary to form the secondary, tertiary, and quaternary structure of the protein of interest. The software looks at certain parameters encoded by the primary sequence to generate the structural model. These parameters are referred to as “energy terms,” and primarily include electrostatic potentials, hydrophobic potentials, solvent accessible surfaces, and hydrogen bonding. Secondary energy terms include van der Waals potentials. Biological molecules form the structures that minimize the energy terms in a cumulative fashion. The computer program is therefore using these terms encoded by the primary structure or amino acid sequence to create the secondary structural model.

The tertiary structure of the protein encoded by the secondary structure is then formed on the basis of the energy terms of the secondary structure. The user at this point can enter additional variables such as whether the protein is membrane bound or soluble, its location in the body, and its cellular location, e.g., cytoplasmic, surface, or nuclear. These variables along with the energy terms of the secondary structure are used to form the model of the tertiary structure. In modeling the tertiary structure, the computer program matches hydrophobic faces of secondary structure with like, and hydrophilic faces of secondary structure with like.

Once the structure has been generated, potential ligand binding regions are identified by the computer system. Three-dimensional structures for potential ligands are generated by entering amino acid or nucleotide sequences or chemical formulas of compounds, as described above. The three-dimensional structure of the potential ligand is then compared to that of a cytokine receptor of interest to identify binding sites of the cytokine receptor. Binding affinity between the cytokine receptor of interest and ligands is determined using energy terms to determine which ligands have an enhanced probability of binding to the receptor.

### VIII. GENE DELIVERY SYSTEMS

In certain aspects, the present invention provides methods for the treatment of a neuroimmune-based psychiatric disorder by administering a therapeutically effective amount of a nucleic acid encoding one or more cytokine receptors of interest as described herein. In some embodiments, the nucleic acid is incorporated into a vector such as a bacterial or viral vector. Examples of vectors used for such purposes include expression plasmids capable of directing the expression of the nucleic acid in a target cell. In some instances, the vector is a viral vector system wherein the nucleic acid is incorporated into a viral genome that is capable of transfecting a target cell. In particular embodiments, the nucleic acid can be operably linked to expression and control sequences that can direct expression of the cytokine receptor in the desired target host cells. Thus, the expression of the nucleic acid under appropriate conditions can be achieved in the target cell.

Viral vector systems useful in the expression of the nucleic acid include, but are not limited to, naturally-occurring or recombinant viral vector systems. Depending upon the particular application, suitable viral vectors include replication competent, replication deficient, and conditionally replicating viral vectors. For example, viral vectors can be derived from the genome of human or bovine adenoviruses, vaccinia virus, herpes virus, adeno-associated virus, minute virus of mice (MVM), HIV, sindbis virus, and retroviruses (including but not limited to Rous sarcoma virus), and MoMLV. Typically, the genes of interest are inserted into such vectors to allow packaging of the gene construct, typically with accompanying viral DNA, followed by infection of a sensitive host cell and expression of the gene of interest.

As used herein, the term “gene delivery system” refers to any means for the delivery of a nucleic acid to a target cell. In some embodiments, nucleic acids are conjugated to a cell receptor ligand for facilitated uptake (e.g., invagination of coated pits and internalization of the endosome) through an appropriate linking moiety, such as a DNA linking moiety (see, e.g., Wu et al., J. Biol. Chem., 263:14621-14624 (1988); PCT Publication No. WO 92/06180). For example, nucleic acids can be linked through a polylysine moiety to asialo-oromucocid, which is a ligand for the asialoglycoprotein receptor of hepatocytes.

Similarly, viral envelopes used for packaging gene constructs that include the nucleic acid can be modified by the addition of receptor ligands or antibodies specific for a receptor to permit receptor-mediated endocytosis into specific cells (see, e.g., PCT Publication Nos. WO 93/20221, WO 93/14188, and WO 94/06923). In some embodiments, the DNA constructs are linked to viral proteins, such as adenovirus particles, to facilitate endocytosis (Curiel et al., Proc. Natl. Acad. Sci. U.S.A., 88:8850-8854 (1991)).

Retroviral vectors are also useful for introducing the nucleic acid into target cells or organisms. Retroviral vectors are produced by genetically manipulating retroviruses. The viral genome of retroviruses is RNA. Upon infection, this genomic RNA is reverse transcribed into a DNA copy which is integrated into the chromosomal DNA of transduced cells with a high degree of stability and efficiency. The integrated DNA copy is referred to as a provirus and is inherited by daughter cells as is any other gene. The wild-type retroviral genome and the proviral DNA have three genes: the gag, the pol and the env genes, which are flanked by two long terminal repeat (LTR) sequences. The gag gene encodes the internal structural (nucleocapsid) proteins; the pol gene encodes the RNA directed DNA polymerase (reverse transcriptase); and the env gene encodes viral envelope glycoproteins. The 5′ and 3′ LTRs serve to promote transcription and polyadenylation of virion RNAs. Adjacent to the 5′ LTR are sequences necessary for reverse transcription of the genome (the tRNA primer binding site) and for efficient encapsulation of viral RNA into particles (the Psi site) (see, Mulligan, In: Experimental Manipulation of Gene Expression, Inouye (ed), 155-173 (1983); Mann et al., Cell 33:153-159 (1983); Cone and Mulligan, Proceedings of the National Academy of Sciences, U.S.A., 81:6349-6353 (1984)).

The design of retroviral vectors is well known to those of ordinary skill in the art. In brief, if the sequences necessary for encapsidation (or packaging of retroviral RNA into infectious virions) are missing from the viral genome, the result is a cis-acting defect which prevents encapsidation of genomic RNA. However, the resulting mutant is still capable of directing the synthesis of all virion proteins. Retroviral genomes from which these sequences have been deleted, as well as cell lines containing the mutant genome stably integrated into the chromosome are well known in the art and are used to construct retroviral vectors. Preparation of retroviral vectors and their uses are described in many publications including, e.g., European Patent No. 0178220; U.S. Pat. No. 4,405,712; Gilboa, Biotechniques, 4:504-512 (1986); Mann et al., Cell, 33:153-159 (1983); Cone and Mulligan, Proc. Natl. Acad. Sci. USA, 81:6349-6353 (1984); Eglitis et al., Biotechniques 6:608-614 (1988); Miller et al., Biotechniques, 7:981-990 (1989); and PCT Publication No. WO 92/07943.

The retroviral vector particles are prepared by recombinantly inserting the desired nucleotide sequence into a retrovirus vector and packaging the vector with retroviral capsid proteins by use of a packaging cell line. The resultant retroviral vector particle is incapable of replication in the host cell but is capable of integrating into the host cell genome as a proviral sequence containing the desired nucleotide sequence. As a result, the individual is capable of producing, for example, a cytokine receptor of interest and thus restore the cells to a normal phenotype.

Packaging cell lines that are used to prepare the retroviral vector particles are typically recombinant mammalian tissue culture cell lines that produce the necessary viral structural proteins required for packaging, but which are incapable of producing infectious virions. The defective retroviral vectors that are used, on the other hand, lack these structural genes but encode the remaining proteins necessary for packaging. To prepare a packaging cell line, one can construct an infectious clone of a desired retrovirus in which the packaging site has been deleted. Cells comprising this construct will express all structural viral proteins, but the introduced DNA will be incapable of being packaged. Alternatively, packaging cell lines can be produced by transforming a cell line with one or more expression plasmids encoding the appropriate core and envelope proteins. In these cells, the gag, pol, and env genes can be derived from the same or different retroviruses.

A number of packaging cell lines suitable for the present invention are also available. Examples of these cell lines include Crip, GPE86, PA317 and PG13 (see Miller et al., J. Virol., 65:2220-2224 (1991)). Examples of other packaging cell lines are described in Cone and Mulligan. Proc. Natl. Acad. Sci. USA, 81:6349-6353 (1984); Danos and Mulligan. Proc. Natl. Acad. Sci. USA, 85:6460-6464 (1988); Eglitis et al. (1988), supra; and Miller (1989), supra.

Packaging cell lines capable of producing retroviral vector particles with chimeric envelope proteins may be used. Alternatively, amphotropic or xenotropic envelope proteins, such as those produced by PA317 and GPX packaging cell lines may be used to package the retroviral vectors.

In other embodiments, conditional expression systems, such as those typified by the tet-regulated systems and the RU-486 system, can be used (see, e.g., Gossen and Bujard, Proc. Natl. Acad. Sci. USA, 89:5547 (1992); Oligino et al., Gene Ther., 5:491-496 (1998); Wang et al., Gene Ther., 4:432-441 (1997); Neering et al., Blood, 88:1147-1155 (1996); and Rendahl et al., Nat. Biotechnol., 16:757-761 (1998)). These systems impart small molecule control on the expression of the target gene(s) of interest.

In further embodiments, stem cells engineered to express one or more cytokine receptors of interest can be implanted into the brain (e.g., in the frontal cortex).

### IX. ADMINISTRATION AND PHARMACEUTICAL COMPOSITIONS

In certain aspects, the imaging agents (e.g., labeled cytokines, related ligands, etc.) and therapeutic agents (e.g., compounds that modulate cytokine receptor expression, nucleic acids encoding cytokine receptors, etc.) described herein are administered directly to an individual (e.g., a human). Administration is by any of the routes normally used for introducing an agent into contact with a tissue to be imaged or treated and is well known to those of skill in the art. Although more than one route can be used to administer a particular composition, a particular route can often provide a more immediate and more effective reaction than another route.

In some embodiments, the therapeutic agents described herein can be combined with other drugs useful for treating psychiatric disorders or symptoms thereof In some embodiments, the pharmaceutical compositions of the present invention may comprise a modulator of cytokine receptor expression (e.g., a cytokine receptor agonist or a nucleic acid encoding a cytokine receptor) combined with at least one additional compound useful for treating psychiatric disorders or symptoms thereof.

The pharmaceutical compositions of the invention may comprise a pharmaceutically acceptable carrier. In certain aspects, pharmaceutically acceptable carriers are determined in part by the particular composition being administered, as well as by the particular method used to administer the composition. Accordingly, there is a wide variety of suitable formulations of pharmaceutical compositions of the present invention (see, e.g., REMINGTON'S PHARMACEUTICAL SCIENCES, 18TH ED., Mack Publishing Co., Easton, Pa. (1990)).

The pharmaceutical compositions of the invention are administered in a manner compatible with the dosage formulation, and in such amount as will be therapeutically effective or suitable for in vivo imaging. The quantity to be administered depends on a variety of factors including, e.g., the age, body weight, physical activity, and diet of the individual, the psychiatric disorder to be imaged or treated, and the stage or severity of the psychiatric disorder. In certain embodiments, the size of the dose may also be determined by the existence, nature, and extent of any adverse side effects that accompany the administration of a particular imaging agent or therapeutic agent in a particular individual. In general, the dose equivalent of an imaging agent or therapeutic agent is from about 1 ng/kg to about 10 mg/kg for a typical individual.

In certain embodiments, the dose may take the form of solid, semi-solid, lyophilized powder, or liquid dosage forms, such as, for example, tablets, pills, pellets, capsules, powders, solutions, suspensions, emulsions, suppositories, retention enemas, creams, ointments, lotions, gels, aerosols, foams, or the like, preferably in unit dosage forms suitable for simple administration of precise dosages.

In the practice of this invention, the compositions can be administered, for example, intravenously, intracranially, intrathecally, intraspinally, intraperitoneally, intramuscularly, intralesionally, intranasally, subcutaneously, intracerebroventricularly, orally, topically, and/or by inhalation.

As used herein, the term “unit dosage form” refers to physically discrete units suitable as unitary dosages for humans and other mammals, each unit containing a predetermined quantity of an imaging agent or therapeutic agent calculated to produce the desired onset, tolerability, and/or therapeutic effects, in association with a suitable pharmaceutical excipient (e.g., an ampoule). In addition, more concentrated dosage forms may be prepared, from which the more dilute unit dosage forms may then be produced. The more concentrated dosage forms thus will contain substantially more than, e.g., at least 1, 2, 3, 4, 5, 6, 7, 8, 9, 10, or more times the amount of the imaging agent or therapeutic agent.

Methods for preparing such dosage forms are known to those skilled in the art (see, e.g., REMINGTON'S PHARMACEUTICAL SCIENCES, 18TH ED., Mack Publishing Co., Easton, Pa. (1990)). The dosage forms typically include a conventional pharmaceutical carrier or excipient and may additionally include other medicinal agents, carriers, adjuvants, diluents, tissue permeation enhancers, solubilizers, and the like. Appropriate excipients can be tailored to the particular dosage form and route of administration by methods well known in the art (see, e.g., REMINGTON'S PHARMACEUTICAL SCIENCES, supra).

Examples of suitable excipients include, but are not limited to, lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, saline, syrup, methylcellulose, ethylcellulose, hydroxypropylmethylcellulose, and polyacrylic acids such as Carbopols, e.g., Carbopol 941, Carbopol 980, Carbopol 981, etc. The dosage forms can additionally include lubricating agents such as talc, magnesium stearate, and mineral oil; wetting agents; emulsifying agents; suspending agents; preserving agents such as methyl-, ethyl-, and propyl-hydroxy-benzoates (i.e., the parabens); pH adjusting agents such as inorganic and organic acids and bases; sweetening agents; and flavoring agents. The dosage forms may also comprise biodegradable polymer beads, dextran, and cyclodextrin inclusion complexes.

For oral administration, the therapeutically effective dose can be in the form of tablets, capsules, emulsions, suspensions, solutions, syrups, sprays, lozenges, powders, and sustained-release formulations. Suitable excipients for oral administration include pharmaceutical grades of mannitol, lactose, starch, magnesium stearate, sodium saccharine, talcum, cellulose, glucose, gelatin, sucrose, magnesium carbonate, and the like.

In some embodiments, the therapeutically effective dose takes the form of a pill, tablet, or capsule, and thus, the dosage form can contain, along with an imaging agent or therapeutic agent described herein, any of the following: a diluent such as lactose, sucrose, dicalcium phosphate, and the like; a disintegrant such as starch or derivatives thereof; a lubricant such as magnesium stearate and the like; and a binder such a starch, gum acacia, polyvinylpyrrolidone, gelatin, cellulose and derivatives thereof. An imaging agent or therapeutic agent can also be formulated into a suppository disposed, for example, in a polyethylene glycol (PEG) carrier.

Liquid dosage forms can be prepared by dissolving or dispersing an imaging agent or therapeutic agent and optionally one or more pharmaceutically acceptable adjuvants in a carrier such as, for example, aqueous saline (e.g., 0.9% w/v sodium chloride), aqueous dextrose, glycerol, ethanol, and the like, to form a solution or suspension, e.g., for oral, topical, or intravenous administration. An imaging agent or therapeutic agent can also be formulated into a retention enema.

For topical administration, the therapeutically effective dose can be in the form of emulsions, lotions, gels, foams, creams, jellies, solutions, suspensions, ointments, and transdermal patches. For administration by inhalation, an imaging agent or therapeutic agent can be delivered as a dry powder or in liquid form via a nebulizer. Aerosol formulations can be placed into pressurized acceptable propellants such as dichlorodifluoromethane. For parenteral administration, the therapeutically effective dose can be in the form of sterile injectable solutions and sterile packaged powders. Preferably, injectable solutions are formulated at a pH of from about 4.5 to about 7.5.

The therapeutically effective dose can also be provided in a lyophilized form. Such dosage forms may include a buffer, e.g., bicarbonate, for reconstitution prior to administration, or the buffer may be included in the lyophilized dosage form for reconstitution with, e.g., water. The lyophilized dosage form may further comprise a suitable vasoconstrictor, e.g., epinephrine. The lyophilized dosage form can be provided in a syringe, optionally packaged in combination with the buffer for reconstitution, such that the reconstituted dosage form can be immediately administered to an individual.

### X. EXAMPLE

The following example is offered to illustrate, but not to limit, the claimed invention.

### Example 1

### Targeting Cytokine Receptors in the Brain for the Development of Novel Diagnostic Tools and Therapies for Neuroimmune-Based Psychiatric Disorders

This example describes the discovery of a specific subset of cytokine receptors that are significantly altered in the frontal cortex of maternal immune activation (MIA) offspring, relative to control offspring, at multiple ages throughout postnatal development in both mouse and non-human primate (NHP) animal models. In certain aspects, the present invention targets one or more of these commonly dysregulated cytokine receptors to identify those individuals with neuroimmune-based psychiatric disorders and to screen libraries of compounds to identify therapeutic agents against these cytokine receptors for the treatment of neuroimmune-based psychiatric disorders.

### Methods

Pregnant C56BL/6 mice at gestational day (GD) 12.5 were injected intraperitoneally with fresh poly (I:C) dsRNA at 20 mg/kg, or vehicle control (sterile 0.9% saline). Animals were weighed to confirm sickness behavior. Frontal cortex was dissected from 10 MIA and 10 saline offspring at each age: P0, P7, P14, P30 and P60 and placed immediately in RNAlater. Offspring were from at least 3 different litters for each age.

Pregnant rhesus monkeys were injected with 0.25 mg/kg of a modified form of the viral mimic, synthetic double-stranded RNA (polyinosinic:polycytidylic acid stabilized with poly-L-lysine) poly (I:C) or saline via intravenous injection on gestational days 43, 44 and 46 or 100, 101 and 103. Animals were sacrificed and perfused at 3-4 years of age. Tissue was frozen and RNA was isolated from the left hemisphere of DLPFC.

RNA was isolated from mouse and monkey samples using RNeasy Mini Kit (Qiagen) and cDNA made using RT² First Strand Kit (SA Biosciences). Custom 96-well RT² Profiler PCR Arrays with 23 candidate genes and 2 controls were purchased from SA Biosciences. All samples were run in duplicate with a control sample on every plate. Samples were run on a Bio-Rad iCycler and analyzed using RT² Profiler PCR Array Data Analysis version 3.5 (SA Biosciences).

Data was subjected to two levels of significance: fold regulation and Wilcoxon-Mann-Whitney test. Candidate genes were chosen that showed greater than 2-fold change in comparison to controls and p≦0.05.

The fold changes were calculated based on age and sex-matched control offspring (i.e., non-MIA offspring from animals injected with saline). The transcript levels were normalized to housekeeping genes that were assessed for every sample and then the fold differences for the candidate genes were analyzed for the MIA offspring relative to the control offspring.

### Results

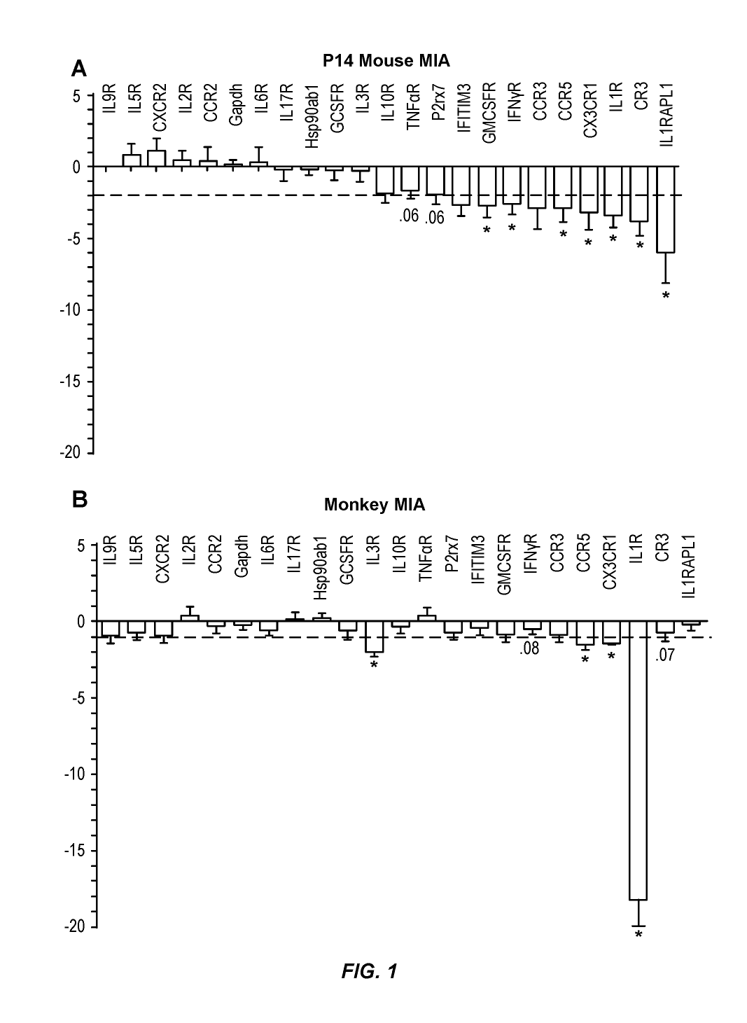

FIG. 1 illustrates the results of gene expression profiling of the 23 candidate genes from the mouse and monkey RNA samples. The time-point in the mouse MIA model that is most similar to changes in the MIA NHP frontal cortex is postnatal day 14 (P14). At this age, there were 7 cytokine receptors whose expression was altered in a way that met stringent criteria for selection as candidate targets, which was a greater than 2-fold change and statistical significance with p<0.05, using a Wilcoxon-Mann-Whitney test. The cytokine receptors that met these criteria in the frontal cortex in the mouse MIA model were IL1RAPL1, CCR5, Cx3CR1, GMCSFR, CR3, IL1R, and IFNγR. There were 4 cytokine receptors that were altered in the dorsolateral prefrontal cortex (DLPFC) of the NHP MIA model by more than 1-fold and met statistical significance of p<0.05 (i.e., IL3R, CCR5, Cx3CR1, and IL-1R). CR3 was also altered by more than 1-fold and almost met significnace (p<0.07). Remarkably, 4 of these receptors, i.e., CR3, CCR5, Cx3CR1, and IL1R, overlapped with those that were altered in the mouse MIA brain. The fifth, IL3R, is known to have largely overlapping functions with GM-CSFR, which was altered in the mouse MIA brain.

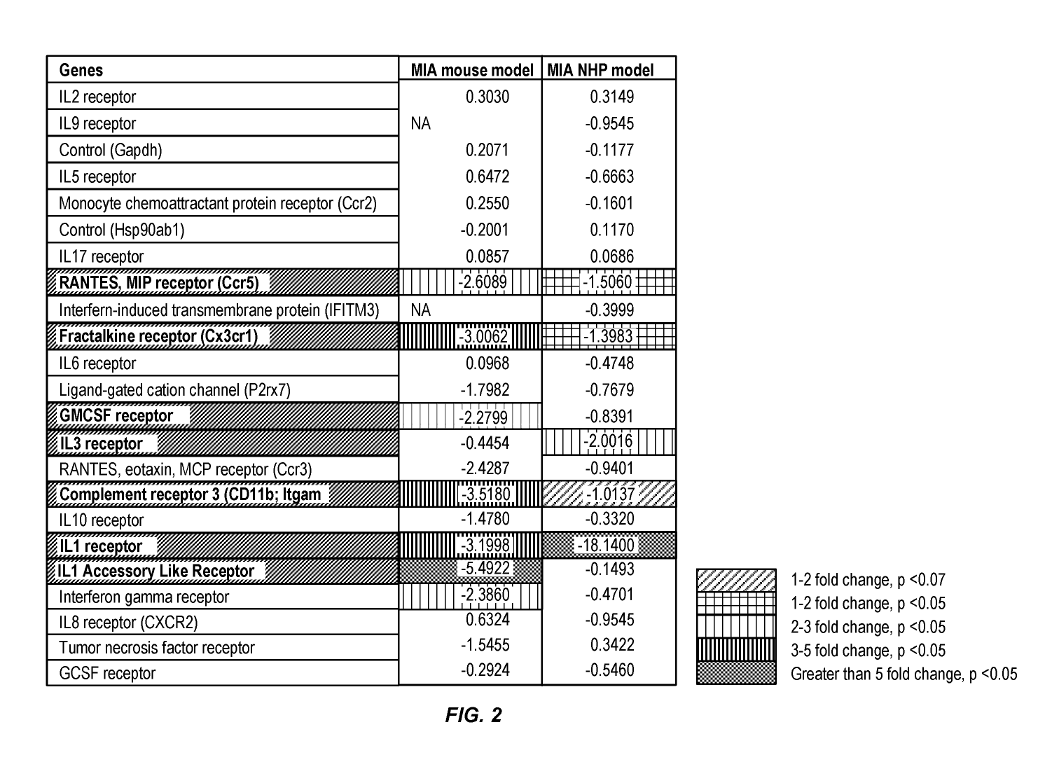

FIG. 2 provides a summary of the MIA-induced changes in cytokine receptors in the frontal cortex in both mouse and NHP offspring. Values representing changes shown in the graphs above are indicated in each box. All of the changes are decreases in the NHP brain and in the mouse brain at P14. Cytokine receptors that meet the criteria of 2-fold changes or greater and p<0.05 for the mouse model and of a 1-2 fold change and p<0.05 for the NHP model are highlighted in shades of gray corresponding to the magnitude of change, indicated in the legend. Cytokine receptors in gray are the candidate receptors targeted for the development of novel diagnostic tools and novel therapies as described herein.

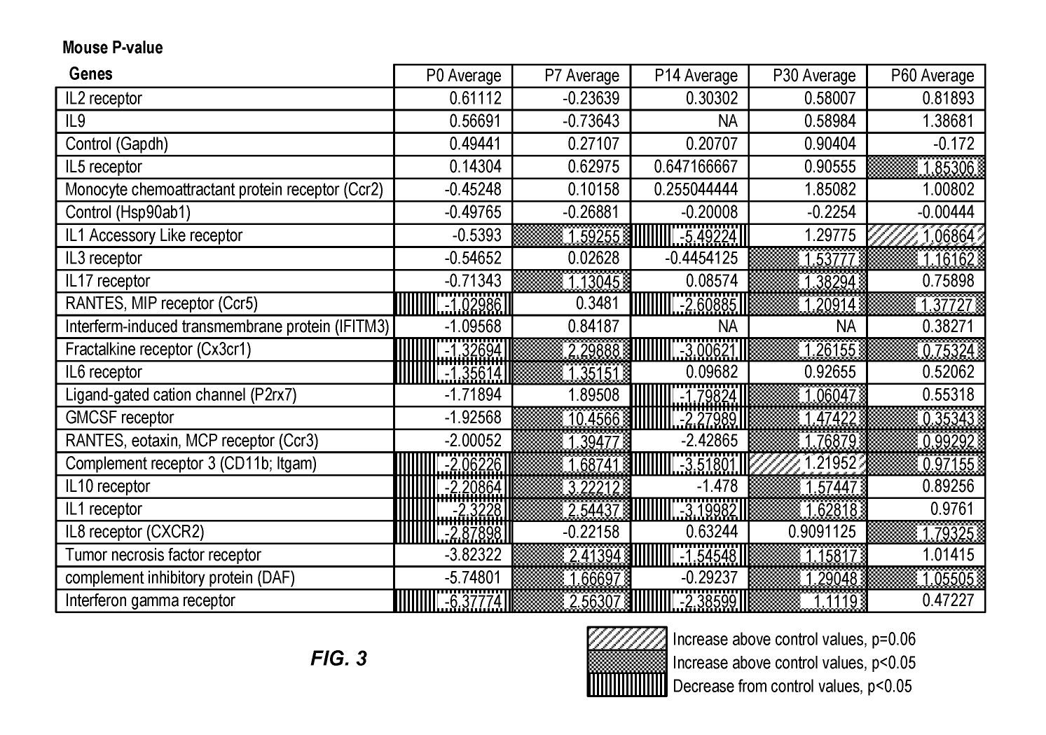

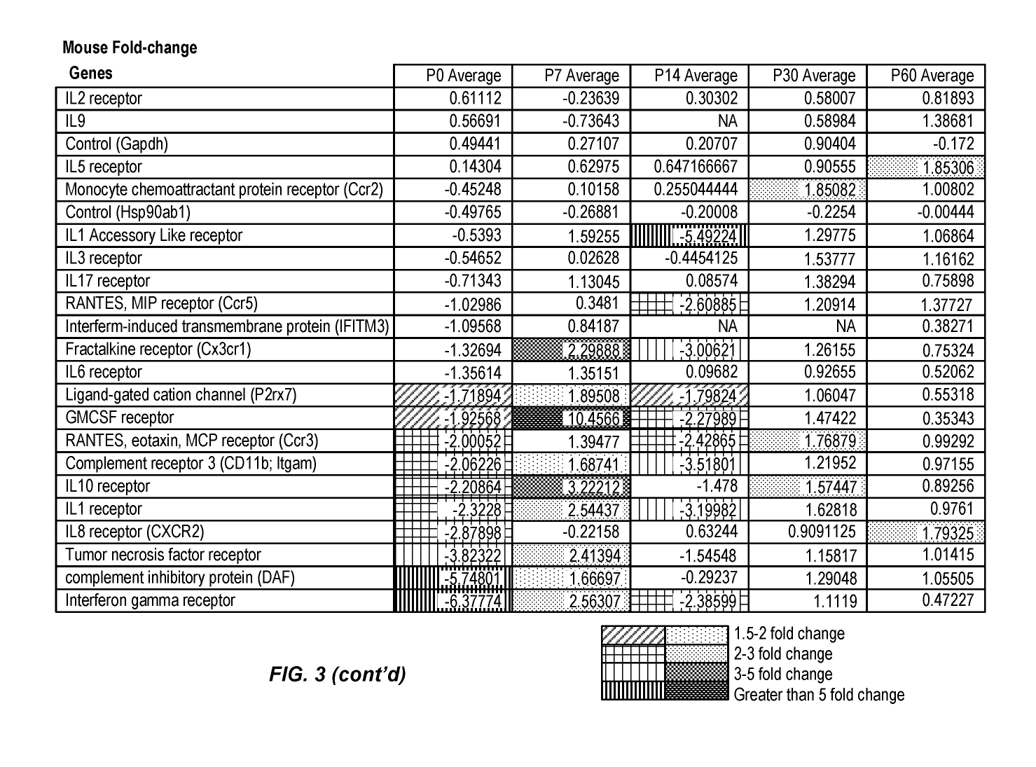

FIG. 3 provides a summary of changes in cytokine receptors in the frontal cortex of MIA offspring from the mouse model at 5 postnatal ages. Cytokine receptors were altered in the brains of MIA offspring throughout postnatal development. P14 was the age used to select target genes because that was the age at which the changes were most similar to those in the NHP brain.

As such, in certain aspects, the present invention targets one or a plurality (i.e., at least two, three, four, five, or six) of the commonly altered cytokine receptors (i.e., CR3, CCR5, Cx3CR1, IL1R, IL3R, and/or GM-CSFR) for: (1) the development of novel diagnostic tools, including PET tracers, to identify those individuals with neuroimmune-based psychiatric illnesses; (2) the creation of high-throughput screens to test the effectiveness of therapeutic agents in the MIA mouse and NHP models; and (3) the development of novel therapeutic agents against these specific cytokine receptors for the treatment of neuroimmune-based psychiatric illnesses.

Although the foregoing invention has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference.

1. A method for determining whether an individual has or is at risk of developing a neuroimmune-based psychiatric disorder, the method comprising:
(a) detecting in a biological sample from the individual the expression level of one or more cytokine receptors selected from the group consisting of CR3, CCR5, Cx3CR1, IL1R, IL3R, GM-C SFR, and combinations thereof;
(b) comparing the expression level of the one or more cytokine receptors detected in the biological sample to a control expression level of the one or more cytokine receptors; and
(c) determining that the individual has or is at risk of developing a neuroimmune-based psychiatric disorder when the expression level of the one or more cytokine receptors detected in the biological sample is decreased compared to a control expression level of the one or more cytokine receptors.

2. The method of claim 1, wherein the neuroimmune-based psychiatric disorder is selected from the group consisting of schizophrenia, autism spectrum disorder, major depressive disorder, and bipolar disorder.

3. The method of claim 1, wherein the individual is a human.

4. The method of claims 1, wherein the expression level of one or more cytokine receptors selected from the group consisting of CR3, CCR5, Cx3CR1, and IL1R is detected.

5. The method of claim 4, wherein the expression level of two, three, or four of the cytokine receptors is detected.

6. The method of claims 1, wherein the expression level is the mRNA level of the one or more cytokine receptors.

7. The method of claim 1, wherein the biological sample is a whole blood, serum, plasma, saliva, urine, cerebrospinal fluid, amniotic fluid, nipple aspirate, or tissue sample.

8. The method of claim 7, wherein the tissue sample is brain tissue.

9. The method of claim 1, wherein the control expression level is the expression level of the one or more cytokine receptors in an individual or a population of individuals without the neuroimmune-based psychiatric disorder.

10. The method of claim 1, wherein the expression level of the one or more cytokine receptors detected in the biological sample is decreased by more than 1-fold compared to the control expression level of the one or more cytokine receptors.

11. The method of claim 1, wherein the method further comprises detecting in the biological sample the expression level of one or more additional cytokine receptors selected from the group consisting of IL1RAPL1, IFNγR, and combinations thereof.

12. A method for the in vivo imaging of brain tissue for determining whether an individual has or is at risk of developing a neuroimmune-based psychiatric disorder, the method comprising:
(a) administering to the individual one or more imaging agents comprising one or more ligands that bind to one or more cytokine receptors selected from the group consisting of CR3, CCR5, Cx3CR1, IL1R, IL3R, GM-CSFR, and combinations thereof, wherein a detectable moiety is attached to the one or more ligands; and
(b) detecting the one or more imaging agents in brain tissue of the individual, wherein the individual has or is at risk of developing a neuroimmune-based psychiatric disorder when the level of the one or more imaging agents detected in the frontal cortex is less than the level of the one or more imaging agents detected in the frontal cortex of an individual or a population of individuals without the neuroimmune-based psychiatric disorder.

13. The method of claim 12, wherein the neuroimmune-based psychiatric disorder is selected from the group consisting of schizophrenia, autism spectrum disorder, major depressive disorder, and bipolar disorder.

14. The method of claim 12, wherein the individual is a human.

15. The method of claim 12, wherein the frontal cortex is the dorsolateral prefrontal cortex (DLPFC).

16. The method of claim 12, wherein the one or more ligands comprises one or more cytokines.

17. The method of claim 16, wherein the one or more cytokines is selected from the group consisting of IL-1β, RANTES, MIP, CCL3L1, GM-CSF, IL-3, CX3CL1, complement component 3b (iC3b), fragments thereof, and combinations thereof.

18. (canceled)

19. (canceled)

20. (canceled)

21. A method for identifying a compound for treating a neuroimmune-based psychiatric disorder, the method comprising:
(a) contacting the compound with one or more cytokine receptors selected from the group consisting of CR3, CCR5, Cx3CR1, IL1R, IL3R, GM-CSFR, and combinations thereof; and
(b) determining whether the compound increases the expression level or activity of the one or more cytokine receptors, thereby identifying a compound for treating a neuroimmune-based psychiatric disorder.

22. (canceled)

23. (canceled)

24. (canceled)

25. (canceled)

26. A method for treating a neuroimmune-based psychiatric disorder in an individual in need thereof, the method comprising:
(a) administering to the individual a therapeutically effective amount of a compound identified using the method of claim 21.

27. (canceled)

28. A method for treating a neuroimmune-based psychiatric disorder in an individual in need thereof, the method comprising:
(a) administering to the individual a therapeutically effective amount of a nucleic acid encoding one or more cytokine receptors selected from the group consisting of CR3, CCR5, Cx3CR1, IL1R, IL3R, GM-CSFR, and combinations thereof.

29. (canceled)

30. (canceled)

31. (canceled)
